(19) United States
(12) Patent Application Publication
(10) Pub. No.: US 2008/0226605 A1
(43) Pub. Date: Sep. 18, 2008

(54) Methods and Compositions Facilitating Entry of Compounds Into Cells

(76) Inventors: Dev Priya Arya, Greenville, SC (US); Carlo V. Catapano, Briones/Minusio (CH)

Correspondence Address: Ballard Spahr Andrews & Ingersoll, LLP, SUITE 1000, 999 PEACHTREE STREET, ATLANTA, GA, 30309-3915, US

(21) Appl. No.: 11/914,683

(22) PCT Filed: May 19, 2006
(86) PCT No.: PCT/US06/19487

§ 371 Date: May 16, 2008

Related U.S. Application Data
(60) Provisional application No. 60/682,496, filed on May 19, 2005.

(51) Int. Cl. A61K 31/7088; C12N 5/08; A61K 47/26; A61P 31/00; C12N 15/88

(52) U.S. Cl. 424/93.7; 435/366; 435/458; 514/777; 514/44

(57) ABSTRACT

Disclosed are methods and compositions for facilitating entry of compounds to cells. In some forms, the compositions comprise one or more aminoglycosides and one or more lipids. The disclosed compositions can also comprise one or more compounds or compositions. It was discovered that the disclosed compositions increase the efficiency of delivery of compounds into cells. The disclosed compositions and methods increase both delivery into cells and the activity of compounds once delivered into cells. For example, the disclosed methods and compositions can be used to deliver nucleic acids to cells and to thereby increase the activity of such nucleic acids delivered to cells. The disclosed compositions can be used to deliver compounds and compositions to cells in vitro, ex vivo and in vivo. Delivery can be, for example, non-specific, non-directed, non-targeted, specific, directed or targeted.

### CROSS-REFERENCE TO RELATED APPLICATIONS

This application claims benefit of U.S. Provisional Application No. 60/682,496, filed May 19, 2005, which is hereby incorporated herein by reference in its entirety.

### STATEMENT REGARDING FEDERALLY SPONSORED RESEARCH

This invention was made with government support under Grant CHE/MCB-0134792 awarded by NSF. The government has certain rights in the invention

### FIELD OF THE INVENTION

The disclosed invention is generally in the field of delivery and entry of compounds to cells and specifically in the area of lipid-based entry of compounds to cells.

### BACKGROUND OF THE INVENTION

DNA delivery, especially by non-viral means (i.e., transfection), has become an important research tool to understand gene structure, regulation and function (Luo and Saltzman, Nat. Biotechnol. 2000, 18, 33; Vijayanathan et al., Biochemistry 2002, 41, 14085). Efficient transfection of foreign DNA in cells, however, remains still a difficult objective to achieve. Presently, cationic lipid-based systems are probably the most commonly used methods of DNA delivery, are relatively non-toxic and have been used occasionally in humans. However, transfection efficiency with these systems remains low and less efficient when compared to viral based DNA delivery system. The overall efficiency of transfection in the case of transgene expression vectors depends on both the efficiency of DNA delivery into cells (uptake) and the efficiency of transgene expression, which is determined by the fraction of vector molecules that enter the nucleus and undergo transcription. A similar problem exists with oligonucleotides, like triplex forming oligonucleotides (TFOs), that need to enter the cell nucleus in order to interact with their intracellular target, the chromosomal DNA. Thus, unlike antisense oligonucleotides, intranuclear, delivery of expression vectors and TFOs is an essential and often limiting step for their biological activity.

### BRIEF SUMMARY OF THE INVENTION

Disclosed are methods and compositions for facilitating entry of compounds to cells. In some forms, the compositions comprise one or more aminoglycosides and one or more lipids. The disclosed compositions can also comprise one or more compounds or compositions. It was discovered that the disclosed compositions increase the efficiency of delivery of compounds into cells. The disclosed compositions and methods increase both delivery into cells and the activity of compounds once delivered into cells. For example, the disclosed methods and compositions can be used to deliver nucleic acids to cells and to thereby increase the activity of such nucleic acids delivered to cells. The disclosed compositions can be used to deliver compounds and compositions to cells in vitro, ex vivo and in vivo. Delivery can be, for example, non-specific, non-directed, non-targeted, specific, directed or targeted. In some forms of the disclosed methods, the compounds and compositions can be delivered into cells by bringing into contact the disclosed compositions and cells.

The disclosed compositions comprise one or more aminoglycosides and one or more lipids. The aminoglycosides can be, for example, aminoglycoside antibiotics, such as neomycin, or derivatives thereof. The aminoglycosides can also be non-antibiotic aminoglycosides. Useful aminoglycosides can interact with nucleic acids. Particularly useful aminoglycosides can be aminoglycosides that can interact with nucleic acids in the same manner as aminoglycoside antibiotics interact with nucleic acids. The amino glycosides can have any number of subunits that allow delivery of a compound or composition to a cell. Useful numbers of subunits include, for example, two, three, four, five, six, and seven subunits. Useful subunits can be linear or circular. The amino glycosides can be linear, branched or circular chains of subunits.

The lipids can be, for example, cationic lipids, such as 1,2-dioleoyl-3-trimethyl-ammonium-propane (DOTAP). The lipids can have, for example, one, two, three or four fatty acid chains. The fatty acid chains can have any suitable length. The fatty acid chains can be saturated or unsaturated. The fatty acid chains can be, for example, monounsaturated, di-unsaturated, or tri-unsaturated. Lipids with multiple fatty acids chains can have any combination of fatty acid chains (that is, the fatty acid chains can be homomeric or heteromeric). The lipids can be pharmaceutically acceptable lipids. Useful lipids include lipids that can be metabolized by the target cells.

The disclosed compositions can have any ratio of aminoglycosides and lipids that allows delivery of a compound or composition to a cell. Useful ratios of aminoglycosides and lipids are those that optimize delivery of a compound or composition of interest to a cell of interest. Such ratios can be determined using techniques disclosed herein (see the Example). Such ratios can also be determined using the disclosed compositions in any delivery method of interest and determining the efficiency of delivery.

The disclosed compositions can have any ratio of aminoglycosides and the compound or composition to be delivered that allows delivery of the compound or composition to a cell. Useful ratios of aminoglycosides and compounds or compositions are those that optimize delivery of a compound or composition of interest to a cell of interest. Such ratios can be determined using techniques disclosed herein (see the Example). Such ratios can also be determined using the disclosed compositions in any delivery method of interest and determining the efficiency of delivery.

The disclosed compositions can have any ratio of lipids and the compound or composition to be delivered that allows delivery of the compound or composition to a cell. Useful ratios of lipids and compounds or compositions are those that optimize delivery of a compound or composition of interest to a cell of interest. Such ratios can be determined using techniques disclosed herein (see the Example). Such ratios can also be determined using the disclosed compositions in any delivery method of interest and determining the efficiency of delivery.

The disclosed compositions can be used in the disclosed methods as well as any known method of delivering compounds and compositions to cells. The disclosed compositions and methods are useful for delivering nucleic acids. Any nucleic acid of interest can be delivered. For example, vectors, genes, expression cassettes, transposons, promoters, enhancers, coding regions, antisense nucleic acids, triplex-forming nucleic acids, short interfering RNA, messenger RNA, ribozymes, and catalytic nucleic acids can be delivered using the disclosed compositions and methods. The disclosed compositions and methods are useful for delivering negatively charged compounds.

Also disclosed are mixtures comprising the disclosed compositions and one or more cells. Also disclosed are sets or populations of cells that have been transfected using the disclosed compositions. For example, disclosed are sets or populations of cells exposed to the disclosed compositions where 10% or more, 20% or more, 25% or more, 30% or more, 40% or more, or 50% or more of the cells are effectively transfected. Also disclosed are sets or populations of cells to which compoumds or compositions have been delivered using the disclosed compositions. For example, disclosed are sets or populations of cells exposed to the disclosed compositions where the compound or composition has been effectively delivered to 10% or more, 20% or more, 25% or more, 30% or more, 40% or more, or 50% or more of the cells.

Disclosed are methods of delivering compounds and compositions to cells. In some forms of the method, compounds and compositions can be delivered into cells in vitro. In some forms of the method, compounds and compositions can be delivered into cells ex vivo. Such cells can be introduced into or administered to a subject. In some forms of the method, compounds and compositions can be delivered to cells in vivo. This can be accomplished by, for example, administering the disclosed compositions to a subject. Delivery of compounds and compositions into cells can be for any purpose. Generally, a given compound or composition can be delivered into a cell for a purpose related to the compound or composition, which purposes are generally known for a large number of compounds and compositions. For example, delivery of a vector to a cell using the disclosed compositions can be to obtain expression of the vector and/or stable transmission of the vector in progeny of the cell; delivery of a drug to a cell using the disclosed compositions can be to obtain an effect on the physiology of the cell by the drug (and thus an effect on the physiology of a subject if the cell is in or introduced to the subject); delivery of a siRNA or ribozyme to a cell using the disclosed compositions can be to obtain a change in, for example, gene expression or RNA processing by the siRNA or ribozyme. The purpose for delivery can be for any effect that the compound or composition can have or for which it was designed. Myriad compounds and compositions are known and they can be used with the disclosed compositions and methods for their known and expected purposed.

Disclosed are methods of treating subjects by administering the disclosed compositions to the subject. For example, compounds and compositions known, expected or suspected of having useful effects on a subject (such as therapeutic effects) can be used in the disclosed methods to treat subjects. Examples of compounds and compositions useful for this purpose include drugs, nucleic acids, and vectors. Also disclosed are methods of treating subjects by bringing into contact the disclosed compositions and cells and then administering the cells to the subject. Also disclosed are methods of administering compounds and compositions to subjects by administering the disclosed compositions to the subject, where the disclosed composition comprises the compound or composition to be delivered. Delivery can be, for example, non-specific, non-directed, non-targeted, specific, directed or targeted.

Additional advantages of the disclosed methods and compositions will be set forth in part in the description which follows, and in part will be understood from the description, or may be learned by practice of the disclosed method and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A and 1B are graphs of enhanced cellular uptake of luciferase reporter plasmids in the presence of neomycin. In FIG. 1 A, cells were transfected with the pRL-SV40 reporter vector using DOTAP and the indicated concentrations of neomycin. An oligonucleotide was added to the transfection mix as carrier to adjust the total amount of DNA to 200 ng/well. Luciferase activity was determined 24 hours after transfection. In FIG. 1B, DU145 cells were transfected with the pGL3-Ets2 and pRL-SV40 vectors (200 ng/well) using DOTAP or DOTAP combined with neomycin. Luciferase activity was measured 24 h later using the dual-luciferase assay system.

FIG. 2 is a graph of increased transfection efficiency of an EGFP expressing plasmid in the presence of neomycin. DU145 cells were transfected with the pEGFP plasmid (4 μg) using DOTAP, neomycin (5 μM), or both DOTAP and neomycin. Cells were analyzed by flow cytometry after 24 hours. Numbers in the figure represent the percentage of EGFP-positive cells (fluorescence intensity>101) in the samples.

FIG. 3 is a graph of increased uptake of fluorescein-labeled oligonucleotide in the presence of neomycin. DU145 cells were transfected with a fluorescein-labeled phosphorothioate TFO (125 nM) in the presence of DOTAP, neomycin (5 μM), or a combination of DOTAP and neomycin.

FIGS. 4A, 4B and 4C are graphs of transfection efficiency of an EGFP expressing plasmid in the presence of neomycin. DU145 cells were transfected with the pEGFP plasmid (4 μg) using DOTAP, neomycin (5 μM), or both DOTAP and neomycin. Cells were analyzed by flow cytometry after 24 hours. FIG. 4A is a histogram representation of flow cytometry data. FIG. 4B are dot plot representations of flow cytometry data. Numbers in FIG. 4B represent the percentage of EGFP-positive cells (fluorescence intensity>101) in the samples. FIG. 4C is a graph showing a plot of the fold increase in the number of EGFP-positive cells relative to DOTAP alone.

FIGS. 5A, 5B and 5C are graphs of uptake of fluorescein-labeled oligonucleotide in the presence of neomycin. DU145 cells were transfected with a fluorescein-labeled phosphorothioate TFO (125 nM) in the presence of DOTAP, neomycin (5 μM), or a combination of DOTAP and neomycin. FIG. 4A is a histogram of fluorescence signals. FIG. 4B are dot plots of fluorescence signals. FIG. 5C is a graph showing the data as fold increase relative to cells incubated with DOTAP alone. Control samples are non-transfected cells.

FIG. 6 is a diagram of an example of the disclosed compositions and method where the aminoglycoside neomycin is combined with the lipid DOTAP to enhance delivery of DNA to cells.

FIG. 7 is a diagram of an example of the disclosed composition where the aminoglycoside is covalently coupled to the lipid via a linker. Several examples of linking structures are shown.

FIG. 8 is a diagram of Scheme 1 for synthesis of an example of a DOTAP-neomycin conjugate with no linker (CH₂)n where n=0; X═NH; Y═NHCS.

FIG. 9 is a diagram of Scheme 2 for synthesis of an example of a DOTAP-neomycin conjugate with linker (CH₂)n where n=2; X═S; Y═NHCSNH.

FIG. 10 is a diagram of Scheme 3 for linking lipids, steroids, or other functional groups with neomycin or other aminoglycosides.

FIG. 11 a diagram of Scheme 4 for synthesis of an example of a steroid-neomycin conjugate by click chemistry. (i) Di-ter-butyldicarbonate, H₂O, DMF, Et₃N, 60° C., 5 h; (ii) 2,4,6-triisopropylbenzenesulphonyl chloride, pyridine, r.t., 24 h; (iii) NaN₃, aq. DMF; 70° C., 10 h; (iv) 4M HCl in dioxane, dioxane, r.t., 10 min.; (v) CuSO₄, Na ascorbate, r.t., 12 hr, 95%.

FIG. 12 is a diagram of Scheme 5 for synthesis of an example of a DOTAP-neomycin conjugate by click chemistry. (i) Di-ter-butyldicarbonate, H₂O, DMF, Et₃N, 60° C., 5 h; (ii) 2,4,6-triisopropylbenzenesulphonyl chloride, pyridine, r.t., 24 h; (iii) NaN₃, aq. DMF; 70° C., 10 h; (iv) CuSO₄, Na ascorbate, r.t., 12 hr; (v) DMAP, pyridine, r.t., overnight (vi) 4M HCl in dioxane, dioxane, r.t., 10 min.

### DETAILED DESCRIPTION OF THE INVENTION

The disclosed method and compositions can be understood more readily by reference to the following detailed description of particular embodiments and the Example included therein and to the Figures and their previous and following description.

Delivery of oligonucleotides has been a major impediment in the development of nucleic acid based drugs. It is important to develop systems that are highly efficient in DNA delivery and transgene expression and, at the same time, can be safely applied to basic and clinical research settings. It is desirable that delivery systems improve both the efficiency of DNA delivery into cells (uptake) and the efficiency of transgene expression. It has been discovered that compositions comprising aminoglycoside (such as neomycin) and cationic lipid (such as DOTAP) enhance transfection efficiency of nucleic acids (such as reporter plasmids and oligonucleotides) and results in a significant increase in transgene expression. The disclosed compositions and methods represent a new means for delivery of compositions and compounds, such as nucleic acids, into cells. In some forms of the disclosed methods, the compounds and compositions can be delivered into cells by bringing into contact the disclosed compositions and cells.

Disclosed are methods and compositions for facilitating entry of compounds into cells. In some forms, the compositions comprise one or more aminoglycosides and one or more lipids. The disclosed compositions can also comprise one or more compounds or compositions. It was discovered that the disclosed compositions increase the efficiency of delivery of compounds into cells. The disclosed compositions and methods increase both delivery into cells and the activity of compounds once delivered into cells. For example, the disclosed methods and compositions can be used to deliver nucleic acids into cells and to thereby increase the activity of such nucleic acids delivered into cells. The disclosed compositions can be referred to as delivery compositions. This can help distinguish the compositions for facilitating entry of compounds and compositions into cells from compositions that are delivered using the delivery compositions. The disclosed compositions can be used to deliver compounds and compositions into cells in vitro, ex vivo and in vivo. Delivery can be, for example, non-specific, non-directed, non-targeted, specific, directed or targeted. The disclosed compositions can also be delivered to one or more cells.

The disclosed compositions comprise one or more aminoglycosides and one or more lipids. The aminoglycosides can be, for example, aminoglycoside antibiotics, such as neomycin, or derivatives thereof. The aminoglycosides can also be non-antibiotic aminoglycosides. Useful aminoglycosides can interact with nucleic acids. Particularly useful aminoglycosides can be aminoglycosides that can interact with nucleic acids in the same manner as aminoglycoside antibiotics interact with nucleic acids. The amino glycosides can have any number of subunits that allow delivery of a compound or composition into a cell. Useful numbers of subunits include, for example, two, three, four, five, six, and seven subunits. Useful subunits can be linear or circular. The amino glycosides can be linear, branched or circular chains of subunits.

The lipids can be, for example, cationic lipids, such as 1,2-dioleoyl-3-trimethyl-ammonium-propane (DOTAP). The lipids can have, for example, one, two, three or four fatty acid chains. The fatty acid chains can be, for example, 5, 6, 7, 8, 9, 10, 11, 12, 13, 14, 15, 16, 17, 18, 19, 20, 21, 22, 23, 24, 25, 26, 27, 28, 29, and 30 carbons in length. The fatty acid chains can be saturated or unsaturated. The fatty acid chains can be, for example, monounsaturated, di-unsaturated, or tri-unsaturated. Lipids with multiple fatty acids chains can have any combination of fatty acid chains (that is, the fatty acid chains can be homomeric or heteromeric). The lipids can be pharmaceutically acceptable lipids. Useful lipids include lipids that can be metabolized by the target cells.

The disclosed compositions can have any ratio of aminoglycosides and lipids that allows delivery of a compound or composition into a cell. Useful ratios of aminoglycosides and lipids are those that optimize delivery of a compound or composition of interest into a cell of interest. Such ratios can be determined using techniques disclosed herein (see the Example). Such ratios can also be determined using the disclosed compositions in any delivery method of interest and determining the efficiency of delivery. Useful ratios include ratios of about 1:100, 1:90, 1:80, 1:70, 1:60, 1:50, 1:45, 1:40, 1:35, 1:30, 1:25, 1:20, 1:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 60:1, 70:1, 80:1, 90:1, and 100:1 of aminoglycoside to lipid (concentration/concentration).

The disclosed compositions can have any ratio of aminoglycosides and the compound or composition to be delivered that allows delivery of the compound or composition into a cell. Useful ratios of aminoglycosides and compounds or compositions are those that optimize delivery of a compound or composition of interest into a cell of interest. Such ratios can be determined using techniques disclosed herein (see the Example). Such ratios can also be determined using the disclosed compositions in any delivery method of interest and determining the efficiency of delivery. Useful ratios include ratios of about 1:100, 1:90, 1:80, 1:70, 1:60, 1:50, 1:45, 1:40, 1:35, 1:30, 1:25, 1:20, 1:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 60:1, 70:1, 80:1, 90:1, and 100:1 of aminoglycoside to the compound or composition (concentration/concentration).

The disclosed compositions can have any ratio of lipids and the compound or composition to be delivered that allows delivery of the compound or composition into a cell. Useful ratios of lipids and compounds or compositions are those that optimize delivery of a compound or composition of interest into a cell of interest. Such ratios can be determined using techniques disclosed herein (see the Example). Such ratios can also be determined using the disclosed compositions in any delivery method of interest and determining the efficiency of delivery. Useful ratios include ratios of about 1:100, 1:90, 1:80, 1:70, 1:60, 1:50, 1:45, 1:40, 1:35, 1:30, 1:25, 1:20, 1:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 60:1, 70:1, 80:1, 90:1, and 100:1 of lipid to the compound or composition (concentration/concentration).

The disclosed compositions can be used in the disclosed methods as well as any known method of delivering compounds and compositions into cells. The disclosed compositions and methods are useful for delivering nucleic acids. Any nucleic acid of interest can be delivered. For example, vectors, genes, expression cassettes, transposons, promoters, enhancers, coding regions, antisense nucleic acids, triplex-forming nucleic acids, short interfering RNA, messenger RNA, ribozymes, and catalytic nucleic acids can be delivered using the disclosed compositions and methods. The disclosed compositions and methods are useful for delivering negatively charged compounds.

Also disclosed are mixtures comprising the disclosed compositions and one or more cells. Also disclosed are sets or populations of cells that have been transfected using the disclosed compositions. For example, disclosed are sets or populations of cells exposed to the disclosed compositions where 10% or more, 20% or more, 25% or more, 30% or more, 40% or more, or 50% or more of the cells are effectively transfected. By effectively transfected is meant that expression of an expressible nucleic acid delivered with the disclosed composition is detectable in progeny of a given cell. Also disclosed are sets or populations of cells into which compounds or compositions have been delivered using the disclosed compositions. For example, disclosed are sets or populations of cells exposed to the disclosed compositions where the compound or composition has been effectively delivered into 10% or more, 20% or more, 25% or more, 30% or more, 40% or more, or 50% or more of the cells. By effectively delivered is meant that the delivered compound or composition has a detectable effect on a given cell.

Disclosed are methods of delivering compounds and compositions into cells. In some forms of the method, compounds and compositions can be delivered into cells in vitro. In some forms of the method, compounds and compositions can be delivered into cells ex vivo. Such cells can be introduced into or administered to a subject. In some forms of the method, compounds and compositions can be delivered into cells in vivo. This can be accomplished by, for example, administering the disclosed compositions to a subject. Delivery of compounds and compositions into cells can be for any purpose. Generally, a given compound or composition can be delivered into a cell for a purpose related to the compound or composition, which purposes are generally known for a large number of compounds and compositions. For example, delivery of a vector into a cell using the disclosed compositions can be to obtain expression of the vector and/or stable transmission of the vector in progeny of the cell; delivery of a drug into a cell using the disclosed compositions can be to obtain an effect on the physiology of the cell by the drug (and thus an effect on the physiology of a subject if the cell is in or introduced to the subject); delivery of a siRNA or ribozyme into a cell using the disclosed compositions can be to obtain a change in, for example, gene expression or RNA processing by the siRNA or ribozyme. The purpose for delivery can be for any effect that the compound or composition can have or for which it was designed. Myriad compounds and compositions are known and they can be used with the disclosed compositions and methods for their known and expected purposed.

Disclosed are methods of treating subjects by administering the disclosed compositions to the subject. For example, compounds and compositions known, expected or suspected of having useful effects on a subject (such as therapeutic effects) can be used in the disclosed methods to treat subjects. Examples of compounds and compositions useful for this purpose include drugs, nucleic acids, and vectors. Also disclosed are methods of treating subjects by bringing into contact the disclosed compositions and cells and then administering the cells to the subject. Also disclosed are methods of administering compounds and compositions to subjects by administering the disclosed compositions to the subject, where the disclosed composition comprises the compound or composition to be delivered. Delivery can be, for example, non-specific, non-directed, non-targeted, specific, directed or targeted.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if an aminoglycoside is disclosed and discussed and a number of modifications that can be made to a number of molecules including the aminoglycoside are discussed, each and every combination and permutation of aminoglycoside and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of maling and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Aminoglycosides

The disclosed compositions include one or more aminoglycosides. The aminoglycosides can be, for example, aminoglycoside antibiotics, such as neomycin, or derivatives thereof. The aminoglycosides can also be non-antibiotic aminoglycosides. Useful aminoglycosides can interact with nucleic acids. Particularly useful aminoglycosides can be aminoglycosides that can interact with nucleic acids in the same manner as aminoglycoside antibiotics interact with nucleic acids. The amino glycosides can have any number of subunits that allow delivery of a compound or composition into a cell. Useful numbers of subunits include, for example, two, three, four, five, six, and seven subunits. Useful subunits can be linear or circular. The amino glycosides can be linear, branched or circular chains of subunits.

The disclosed compositions can comprise one or more of the same aminoglycoside or one or more different aminoglycosides. An aminoglycoside, as defined herein, is a complex sugar in which two or more aminohexose groups are connected by a glycosidic linkage. In some instances, an aminoglycoside can contain a nucleus or core moiety, for example a streptidine or 2-deoxystreptidine, wherein one or more of the aminohexose groups are linked to the nucleus. Suitable aminoglycosides, which can be used in the disclosed compositions and methods, can have several different aminohexose groups present in the same molecule or, alternatively, an aminoglycoside can have the same aminohexose groups present therein.

Suitable aminohexose groups that can be present in the disclosed aminoglycosides include any hexose where at least one of the hydroxyl groups on the hexose is replaced by an amino group (NH₃) or a protonated amino group (NH₄₊). For example, a suitable aminohexose can be a hexose (i.e., a six carbon carbohydrate) that has one, two, three, or four hydroxyl groups replaced by amino substituents. Such hexoses can be either aldohexoses or ketohexoses and can exist in their cyclic form (e.g., a furanose or pyranose). Some suitable aminoglycosides can contain only aldohexoses where one or more hydroxyl groups have been replaced by an amino group or protonated amino group, only ketohexoses where one or more hydroxyl groups have been replaced by amino groups or protonated amino groups, or one or more such aldohexoses and one or more such ketohexoses. Some specific examples of suitable hexoses include, but are not limited to, D- or L-isomers of allose, altrose, glucose, mannose, glucose, idose, galactose, talose, fructose, psicose, sorbose, and tagatose.

As noted, aminoglycosides that can be used in the disclosed compositions can comprise two or more such aminohexose groups. For example, a suitable aminoglycoside can comprise two, three, four, five, or more than five aminohexose groups. In other examples, a suitable aminoglycoside can comprise at least two, at least three, at least four, or at least five aminohexose groups. The aminohexose groups can be attached to a core moiety. In a still further example, the same aminohexose groups can be present in the aminoglycoside. It yet another example, more than one type of aminohexose group can be present in an aminoglycoside.

In another aspect, suitable aminoglycosides can exhibit antibiotic activity. For example, several aminoglycosides are antibiotics known to bind to the 30S ribosome and inhibit bacterial protein synthesis. They are often used against aerobic Gram-negative bacteria, but also show activity against some Gram-positive bacteria. Further, suitable aminoglycosides can also exhibit DNA binding properties.

Some specific examples of suitable aminoglycosides include, but are not limited to, amikacin (e.g., AMIKIN™), gentamicin (e.g., GARAMYCIN™), hygromycin B, kanamycin (e.g., KANTREX™), neomycin (e.g., MYCIFRADIN™), netilmicin (e.g., NETROMYCIN™), paromomycin (e.g., HUMATIN™), and streptomycin, tobramycin (e.g., TOBI SOLUTION™, TOBRADEX™, NEBCIN™). Also suitable for the disclosed methods and compositions are the sulfate, hydrate, hydrochloride, and free base forms of the aminoglycosides disclosed herein. In one example, the composition can comprise the aminoglycoside neomycin.

Aminoglycosides include modified forms and derivatives of any aminoglycoside structure. For example, one or more amine groups in the disclosed aminoglycosides can be modified or derivatized. As another example, amine and hydroxyl groups can be derivatized with substituted or unsubstituted alkyl groups, substituted or unsubstituted alkene groups, substituted or unsubstituted alkynyl groups, substituted or unsubstituted lipids, or a combination. Examples of useful modified aminoglycosides and useful aminoglycoside modifications are described in Luedtke et al., J. Amer. Chem. Soc. 125:12374-12375 (2003); Kim et al., Biochemistry 43:2373-2383 (2004); and Lesniak et al., Inorganic Chemistry 42(5):1420-1429 (2003).

### B. Lipids

The disclosed compositions include one or more lipids. The lipids can be, for example, cationic lipids, such as 1,2-dioleoyl-3-trimethyl-ammonium-propane (DOTAP). The lipids can have, for example, one, two, three or four fatty acid chains. The fatty acid chains can be, for example, 5, 6, 7, 8, 9, 10, 11, 12, 13, 14, 15, 16, 17, 18, 19, 20, 21, 22, 23, 24, 25, 26, 27, 28, 29, and 30 carbons in length. The fatty acid chains can be saturated or unsaturated. The fatty acid chains can be, for example, monounsaturated, di-unsaturated, or tri-unsaturated. Lipids with multiple fatty acids chains can have any combination of fatty acid chains (that is, the fatty acid chains can be homomeric or heteromeric). The lipids can be pharmaceutically acceptable lipids. Useful lipids include lipids that can be metabolized by the target cells.

In one aspect, the disclosed compositions can comprise a cationic lipid. Suitable cationic lipids, which are disclosed herein, can produce various colloidal systems (e.g., micelles, vesicles, liposomes, etc.) and can interact with polyanions such as DNA and RNA, resulting in various complexes (Felgner et al., Proc. Natl. Acad. Sci. U.S.A. 84:7413-7417 (1987); Felgner et al., Focus 11:21-25 (1989)). It is contemplated that both monovalent and polyvalent cationic lipids can be used in the disclosed compositions and methods. Also, the cationic lipids disclosed herein can be used alone, with a mixture of different cationic lipids, and/or with neutral lipids to form such colloidal systems.

In one example, a suitable cationic lipid can be a derivative of 1-amino-2,3-dihydroxypropane. For example, 1-amino-2,3-dihydroxypropane can be linked to a fatty acid via ester or ether linkages. Examples of suitable fatty acids include, but are not limited to, capric acid (C10), lauric acid (C12), myristic acid (C14), palmitic acid (C16), margaric acid (C17), stearic acid (C18), arachidic acid (C20), behenic acid (C22), lignoceric acid (C24), cerotic acid (C26), montanic acid (C28), and melissic acid (C30), including branched and substituted derivatives thereof such as palmitooleic acid, petroselinic acid, gadolenic acid, vaccenic acid, cetoleic acid, erucic acid, selacholeic acid, ximenic acid, lumequic acid, linolenic acid, linoleic acid, arachidonic acid, adrenic acid, oleic acid, and the like. Also, the 1-amino-2,3-dihydroxypropane can be functionalized at the amino group to result in a quaternary ammonium (i.e., cationic) species. For example, the amino group can be alkylated with a substituted or unsubstituted alkyl group (e.g., methyl, ethyl, propyl, butyl) or cholesterol group. One specific example of such a cationic lipid suitable for use herein is 1,2-dioleoyl-3-N,N,N-trimethylaminopropane chloride (DOTMA). In another specific example, a suitable cationic lipid is 1,2 bis(oleoyloxy)-3-(trimethylammonio) propane (DOTAP).

A related group of compounds, which differ from DOTMA and DOTAP, have one of the methyl groups of the trimethylammonium group replaced by a hydroxyethyl group. Compounds of this type are similar to the Rosenthal Inhibitor of phospholipase A (Rosenthal et al., J. Biol. Chem. 235:2202-2206 (1960), which has stearoyl esters linked to the propylamine core. The dioleoyl analogs of the Rosenthal Inhibitor (RI) are commonly abbreviated as DORI-ether and DORI-ester, depending upon the linkage of the fatty acid moieties to the propylamine core.

Other examples of suitable cationic lipids include, but are not limited to, TC-Chol, dimethyldioctadecylaminonium bromide (DDAB), dioleyldimethylammonium chloride (DODAC), 2,3-dioleoyloxy-N-[2-(spermidine carboxyamido) ethyl]-N,N-dimethyl-1-propaninium trifluoroacetate (DOSPA), carboxylspermylglycine dioctadecylamide (DOGS), diheptadecylamidoglycylspermidine (DHGS). Such cationic lipids are known in the art, and can be used either alone or in combination with other lipids. For example, a DOTMA:DOPE (1:1) formulation is sold under the name LIPOFECTIN™ (GIBCO/BRL: Life Technologies, Inc., Gaithersburg, Md.). DOGS is available commercially as TRANSFECTAM™ (Promega, Madison, Wis.).

Various formulations of cationic lipids have been used to transfect cells in vitro (WO 91/17424; WO 91/16024; U.S. Pat. Nos. 4,897,355; 4,946,787; 5,049,386; and 5,208,036). Cationic lipids have also been used to introduce foreign polynucleotides into frog and rat cells in vivo (Holt et al., Neuron 4:203-214 (1990); Hazinski et al., Am. J. Respr. Cell. Mol. Biol. 4:206-209 (1991)). Useful lipids are described in Martin et al., Current Pharmaceutical Design, 11:375-394 (2005), and Chesnoy and Huang, Ann. Rev. Biophys. Biomol. Struct. 2000 29:27-47 (2000), both of which are hereby incorporated by reference for its description of lipids and their use. Therefore, cationic lipids can be used in the disclosed compositions as pharmaceutical carriers to provide biologically active substances (for example, see WO 91/17424; WO 91/16024; and WO 93/03709).

### C. Delivery Compositions

Delivery compositions (the disclosed compositions) include one or more aminoglycosides and one or more lipids. Any combination of aminoglycosides and lipids can be combined to form a delivery composition. Useful aminoglycosides and lipids for use in the disclosed delivery compositions are described elsewhere herein. The disclosed compositions can have any suitable structure. For example, the disclosed compositions can be mixtures of aminoglycosides and lipids, complexes of aminoglycosides and lipids, conjugates of aminoglycosides and lipid, micelles, lipid bilayers, liposomes, or a combination. Such mixtures, conjugates, micelles, lipid bilayers and liposomes can also comprise one or more compounds and/or one or more compositions to be delivered. For example, conjugates of aminoglycosides, lipids and nucleic acids can be formed and used as delivery compositions.

Aminoglycosides and lipids in delivery compositions can be associated or linked in any suitable manner. For example, aminoglycosides can interact or be linked with lipids non-covalently, ionically, or covalently. Non-covalent interactions can be or any type or combination of types. Thus, for example, aminoglycosides and lipids can interact through polar interactions, charge interactions, van der Waals forces, hydrophobic interactions, or any combination of these. Aminoglycosides can be covalently coupled in any suitable manner, either directly, via a linkage group, or via a linker. In a given delivery composition, different aminoglycosides and different lipids can interact or be linked with each other in different ways. Thus, for example, some of the aminoglycosides can be non-covalently associated with some of the lipids in a delivery composition while other amino glycosides are covalently coupled to other (or the same) lipids.

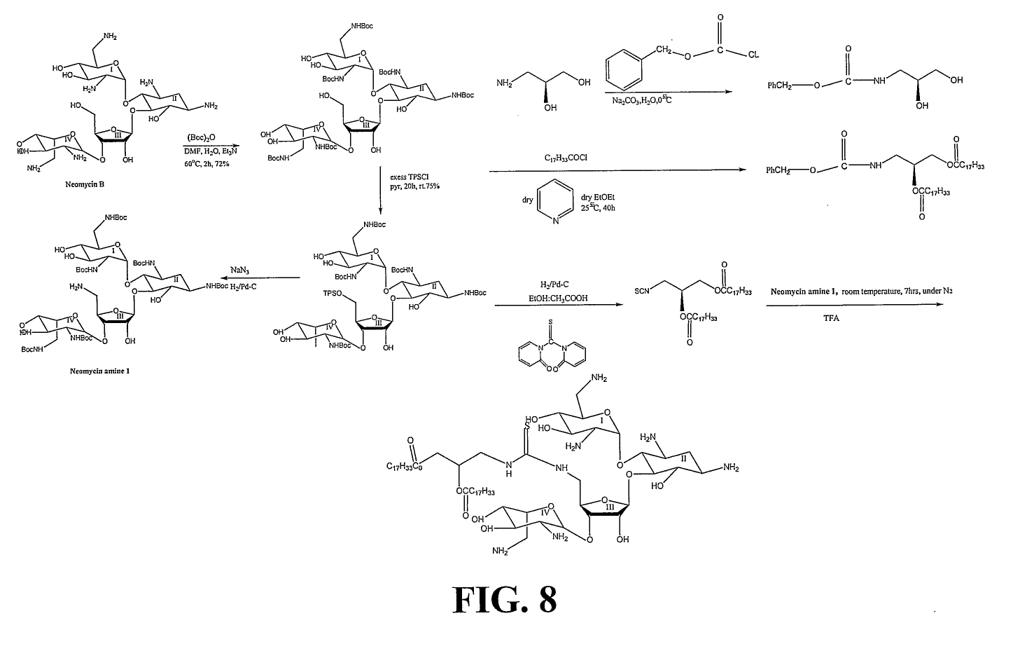

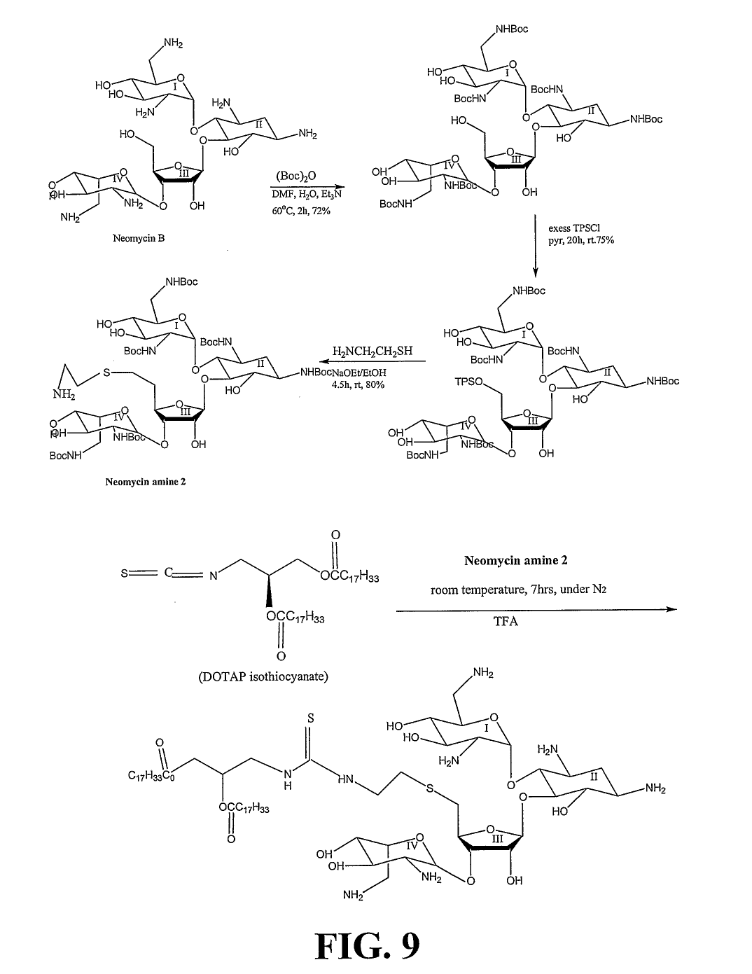

Examples of suitable methods for linking aminoglycosides and lipids, fatty acids, steroids, or other desired structures are provided in FIGS. 8, 9, and 10. Many coupling chemistries are known and can be adapted for use in coupling or linking aminoglycosides and lipids. For example, crosslinking of aminoglycosides to fatty acids, lipids, or steroids can be based on click chemistry. The term “click chemistry” refers to any crosslinking chemistry that is highly favorable under mild conditions and was first coined by Valerie Fokin and K. Barry Sharpless in regards to the triazole-forming reaction between an azide and an alkyne in aqueous environment (Rostovtsev et al., Angew. Cliem. Imt. Ed. 2002, 41, 2596-9). This crosslinking chemistry, which has been used in drug discovery (Lee et al., J. Am. Chem. Soc. 2003, 125, 9588-9; Lewis et al., Angew. Chem. Iit. Ed. 2002, 41, 1053-7; Lewis et al, J. Am. Chem. Soc. 2004, 126, 9152-3), fluorogenic probes (Zhou and Fahuni, J. Am. Chem. Soc. 2004, 126, 8862-3), and cell surface engineering (Link et al., J. Am. Cliem. Soc. 2004, 126, 10598-602; Agard et al., J Am. Chem. Soc. 2004, 126, 15046-7), typically requires the use of copper(I) as a catalyst that has known micromolar toxicity (Arciello et al., Biochem. Biophys. Res. Commun. 2005, 327, 454-9; Smet et al., Hum. Exp. Toxicol. 2003, 22, 89-93; Seth et al., Toxicol. In Vitro 2004, 18, 501-9). In order to reduce the risk of toxicity or inflammation, disclosed herein, in some examples, is the use of catalyst-free click chemistry, which can be accomplished using, for example, electron-deficient alkynes (Li et al., Tetrahedron Lett. 2004, 45, 3143-3146). All of the references disclosed in this paragraph are hereby incorporated by reference at least for their teaching of click chemistry.

Aminoglycosides and lipids can also be coupled via linkers. A linker can be any chain, structure, or region (other than the aminoglycoside or lipid themselves) that links an aminoglycoside and lipid. The linker can have a branched linker structure. Any core or branched structure can form the junction of an aminoglycoside and lipid. An aminoglycoside can interact or be linked with one or more than one lipid. A lipid can interact or be linked with one or more than one aminoglycoside.

The disclosed delivery compositions can have any ratio of aminoglycosides and lipids that allows delivery of a compound or composition into a cell. Useful ratios of aminoglycosides and lipids are those that optimize delivery of a compound or composition of interest into a cell of interest. Such ratios can be determined using techniques disclosed herein (see the Example). Such ratios can also be determined using the disclosed compositions in any delivery method of interest and determining the efficiency of delivery. Useful ratios include ratios of about 1:100, 1:90, 1:80, 1:70, 1:60, 1:50, 1:45, 1:40, 1:35, 1:30, 1:25, 1:20, 1:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 60:1, 70:1, 80:1, 90:1, and 100:1 of aminoglycoside to lipid (concentration/concentration).

The disclosed delivery compositions can have any ratio of aminoglycosides and the compound or composition to be delivered that allows delivery of the compound or composition into a cell. Useful ratios of aminoglycosides and compounds or compositions are those that optimize delivery of a compound or composition of interest into a cell of interest. Such ratios can be determined using techniques disclosed herein (see the Example). Such ratios can also be determined using the disclosed compositions in any delivery method of interest and determining the efficiency of delivery. Useful ratios include ratios of about 1:100, 1:90, 1:80, 1:70, 1:60, 1:50, 1:45, 1:40, 1:35, 1:30, 1:25, 1:20, 1:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 60:1, 70:1, 80:1, 90:1, and 100:1 of aminoglycoside to the compound or composition (concentration/concentration).

The disclosed delivery compositions can have any ratio of lipids and the compound or composition to be delivered that allows delivery of the compound or composition into a cell. Useful ratios of lipids and compounds or compositions are those that optimize delivery of a compound or composition of interest into a cell of interest. Such ratios can be determined using techniques disclosed herein (see the Example). Such ratios can also be determined using the disclosed compositions in any delivery method of interest and determining the efficiency of delivery. Useful ratios include ratios of about 1:100, 1:90, 1:80, 1:70, 1:60, 1:50, 1:45, 1:40, 1:35, 1:30, 1:25, 1:20, 1:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 60:1, 70:1, 80:1, 90:1, and 100:1 of lipid to the compound or composition (concentration/concentration).

The disclosed compositions can be targeted to particular cells or cell types via targeting agents such as antibodies, receptors, or receptor ligands. Many cell components are known that can be used as targets, including, for example, cell surface proteins such as receptors. Targeting can be accomplished by, for example, including a targeting agent in the disclosed composition. For example, known techniques for targeting, for example, drugs, liposomes, particles and the like can be used with the disclosed compositions for targeting.

The disclosed compositions can be administered in vivo or ex vivo. For this purpose, the aminoglycosides and lipids can be pharmaceutically acceptable. By “pharmaceutically acceptable” is meant a material that is not biologically or otherwise undesirable, i.e., the material can be administered to a subject, along with a compound or composition, without causing any undesirable biological effects or interacting in a deleterious manner with any of the other components of the composition in which it is contained. The aminoglycosides and lipids can be selected to minimize any degradation of the active ingredient and to minimize any adverse side effects in the subject, as would be well known to one of skill in the art.

The disclosed compositions can be used in combination with other components. For example, the disclosed compositions can be used in combination with other pharmaceutically acceptable components. An appropriate amount of a pharmaceutically-acceptable salt can be used with the composition to render the formulation isotonic. Examples of the pharmaceutically-acceptable components include, but are not limited to, saline, Ringer's solution and dextrose solution. The pH of the solution is preferably from about 5 to about 8, and more preferably from about 7 to about 7.5. It will be apparent to those persons skilled in the art that certain components can be more preferable depending upon, for instance, the route of administration and concentration of composition being administered.

The disclosed compositions can include carriers, thickeners, diluents, buffers, preservatives, surface active agents and the like in addition to the molecule of choice. The disclosed compositions can also include one or more active ingredients such as antimicrobial agents, anti-inflammatory agents, anesthetics, and the like.

Preparations of the disclosed compositions for parenteral administration can include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like. Parenteral administration of the disclosed composition, if used, is generally characterized by injection. Injectables can be prepared in conventional forms, either as liquid solutions or suspensions, solid forms suitable for solution of suspension in liquid prior to injection, or as emulsions. The disclosed compositions can be in solution, suspension (for example, incorporated into microparticles or liposomes).

Formulations for topical administration can include ointments, lotions, creams, gels, drops, suppositories, sprays, liquids and powders. Conventional pharmaceutical carriers, aqueous, powder or oily bases, thickeners and the like can be used.

Compositions for oral administration include powders or granules, suspensions or solutions in water or non-aqueous media, capsules, sachets, or tablets. Thickeners, flavorings, diluents, emulsifiers, dispersing aids or binders can be used.

### D. Compounds and Compositions

The disclosed compositions can be used to deliver to cells any compounds or compositions of interest. For example, nucleic acids, proteins, peptides, hormones, drugs can be delivered using the disclosed compositions. Nucleic acids are particularly useful compounds for delivery using the disclosed compositions. The disclosed compositions and methods are useful for delivering negatively charged compounds. The disclosed compositions are a general delivery system and so there is no limit to the compounds and compositions that can be delivered. Numerous compounds and compositions are known and the delivery of which to cells would be useful or have useful effects. Such compounds and compositions can be delivered using the disclosed compositions. The compounds and compositions to be delivered can be, for example, antiviral, anti-cancer, and anti-bacterial.

The compounds and compositions to be delivered can be included in the disclosed compositions at any suitable concentration, dosage, or amount. Useful concentrations, dosages, and amounts of compounds and compositions to be delivered are those that, once delivered, will have a desired or intended effect on the cell or population of cells into which the compound or composition is delivered. Effective amounts, concentrations, and dosages of numerous compounds and compositions are known. The increased efficiency of delivery using the disclosed compositions can allow lower amounts, concentrations, and dosages of compounds and compositions to be used.

1. Aminoglycoside Conjugates
Provided herein is a compound comprising the general structure:

R¹-L-R²,
wherein R¹ is a residue of an aminoglycoside;
wherein L is a linker moiety; and
wherein R² is a bilayer transport moiety.

The R¹ residue can be, for example, aminoglycoside antibiotics or derivatives thereof. The aminoglycosides can also be non-antibiotic aminoglycosides. Useful aminoglycosides can interact with nucleic acids. An aminoglycoside, as defined herein, is a complex sugar in which two or more aminohexose groups are collected by a glycosidic linkage. Suitable aminohexose groups that can be present in the disclosed aminoglycosides include any hexose where at least one of the hydroxyl groups on the hexose is replaced by an amino group (NH₃) or a protonated amino group (NH₄⁴⁺). For example, a suitable aminohexose can be a hexose (i.e., a six carbon carbohydrate) that has one, two, three, or four hydroxyl groups replaced by amino substituents. Such hexoses can be either aldohexoses or ketohexoses and can exist in their cyclic form (e.g., a furanose or pyranose). Some suitable aminoglycosides can contain only aldohexoses where one or more hydroxyl groups have been replaced by an amino group or protonated amino group, only ketohexoses where one or more hydroxyl groups have been replaced by amino groups or protonated amino groups, or one or more such aldohexoses and one or more such ketohexoses. Some specific examples of suitable hexoses include, but are not limited to, D- or L-isomers of allose, altrose, glucose, mannose, glucose, idose, galactose, talose, fructose, psicose, sorbose, and tagatose. Thus, R¹ residue can be amikacin (e.g., AMIKIN™), gentamicin (e.g., GARAMYCIN™), hygromycin B, kanamycin (e.g., KANTREX™), neomycin (e.g., MYCIFRADIN™), netilmicin (e.g., NETROMYCIN™), paromomycin (e.g., HUMATIN™), and streptomycin, tobramycin (e.g., TOBI SOLUTION™, TOBRADEX™, NEBCIN™).

The R² bilayer transport moiety can be any lipid-soluble residue capable of crossing a cellular membrane. Thus, the R² moiety can be a lipid. Lipids can be, for example, cationic lipids, such as 1,2-dioleoyl-3-trimethyl-ammonium-propane (DOTAP). The lipids can have, for example, one, two, three or four fatty acid chains. The fatty acid chains can be, for example, 5, 6, 7, 8, 9, 10, 11, 12, 13, 14, 15, 16, 17, 18, 19, 20, 21, 22, 23, 24, 25, 26, 27, 28, 29, and 30 carbons in length. The fatty acid chains can be saturated or unsaturated. The fatty acid chains can be, for example, monounsaturated, di-unsaturated, or tri-unsaturated. Lipids with multiple fatty acids chains can have any combination of fatty acid chains (that is, the fatty acid chains can be homomeric or heteromeric). The lipids can be pharmaceutically acceptable lipids. Useful lipids include lipids that can be metabolized by the target cells.

In one aspect, the R² moiety is a steroid. The main feature, as in all lipids, is the large number of carbon-hydrogens which make steroids non-polar. Steroids include such well known compounds as cholesterol, sex hormones, birth control pills, cortisone, and anabolic steroids. The main feature of steroids is the ring system of three cyclohexanes and one cyclopentane in a fused ring system as shown below.

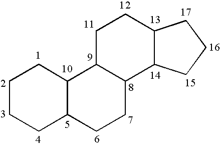

Examples of commercially available steroids that can be linked to aminoglycosides:

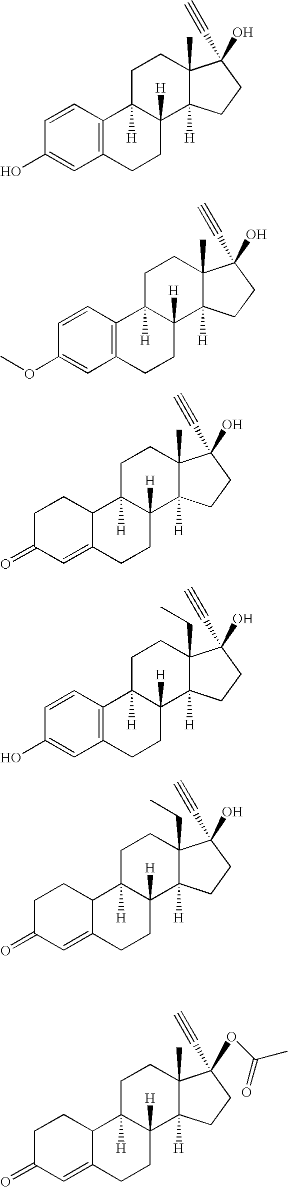

The L residue can comprise any crosslinking moiety suitable for covalently linking R¹ and R². Many coupling chemistries are known and can be adapted for use in coupling or linking aminoglycosides and lipids. For example, crosslinking of aminoglycosides to fatty acids, lipids, or steroids can be based on click chemistry. “Click chemistry” refers to copper catalyzed Huisgen [3+2] cycloadditon reactions. Click Chemistry involves the reaction of an azide and an alkyne to undergo a 1,3 dipolar cycloaddition reaction to yield a 1,2,3-triazole derivatives (Scheme 6). The yield of the reactions is generally very high making them suitable for drug synthesis. The “Click reactions” using Cu (I) catalyst have enabled high regiospecific control (Scheme 7).

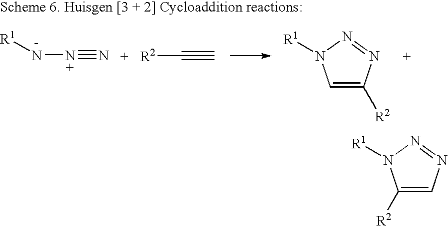

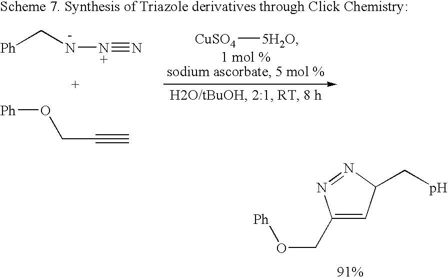

Click Chemistry has been used for a variety of applications in approaches towards the drug discovery, such as the synthesis of neoglycoconjugates, development of HIV protease inhibitors, target guided synthesis under physiological conditions, tagging of live organisms and proteins, activity-based protein profiling and labeling of DNA.

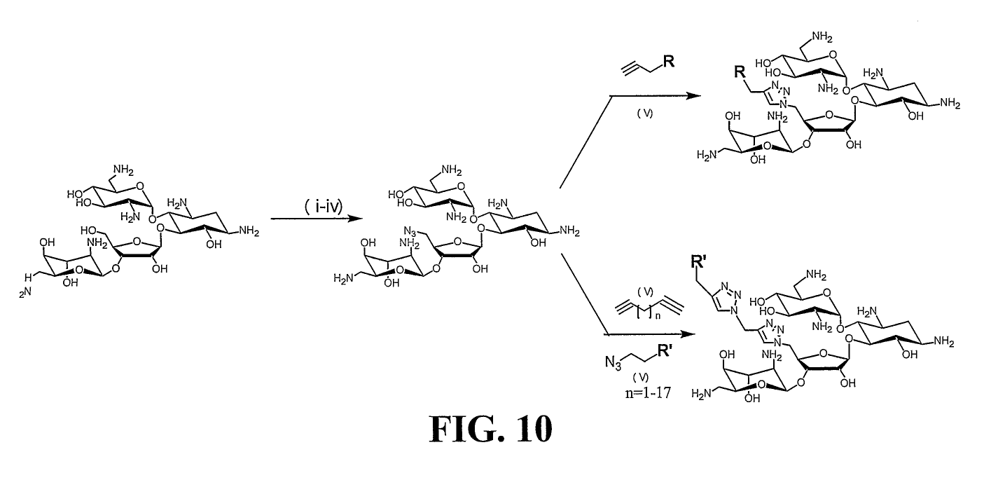

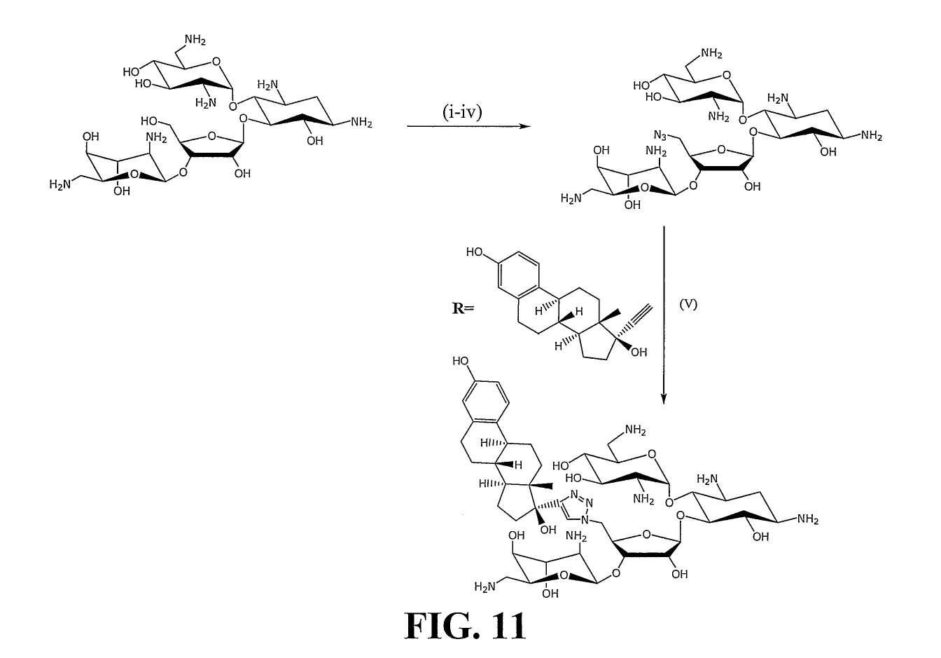

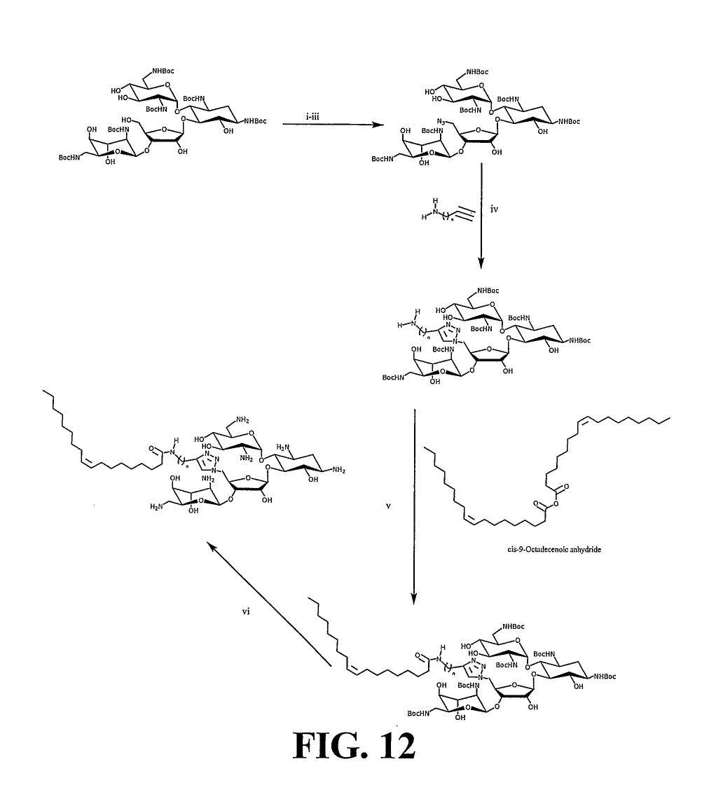

This use of click chemistry has been expanded herein to include aminoglycoside conjugates (FIG. 10). Conversion of neomycin to 5′-azides and 5′-alkynes as shown in FIG. 10 can be used to develop aminoglycoside conjugates using click chemistry. The methodology can be similarly used to link aminoglycosides to lipids, fatty acids and steroids that have been used previously to aid gene transfection. An example of a steroid and lipid chain linked to neomycin is shown in FIGS. 11 and 12, respectively.

2. Nucleic Acids

Nucleic acids can be delivered using the disclosed compositions and methods. Nucleic acids for use in the disclosed compositions include, for example, nucleic acids that encode proteins and peptides of interest and functional nucleic acids. The disclosed nucleic acids can be made up of, for example, nucleotides, nucleotide analogs, or nucleotide substitutes. Non-limiting examples of these and other molecules are discussed herein. It is understood that, for example, when a vector is expressed in a cell, that the expressed mRNA will typically be made up of A, C, G, and U. Likewise, it is understood that if, for example, an antisense molecule is introduced into a cell, it is advantageous that the antisense molecule be made up of nucleotide analogs that reduce the degradation of the antisense molecule in the cellular environment. Patil et al., The AAPS Journal 2005: 7(1) Article 9, describes types and classes of DNA-based therapeutics, which can be delivered using the disclosed compositions. Patil et al. is hereby incorporated by reference for its description of DNA-based therapeutics.

i. Vectors and Expression Sequences

Vectors can be used to express genes and/or to incorporate nucleic acid or genes into the genome of cells. Vectors can have nucleic acid sequences providing functions such a replication sequences, marker or reporter genes, integration sequences, excision sequences, coding sequences, and regulatory sequences, such as promoters, enhancers, silencers, ribosome binding sites, RNA processing sequences, polyadenylation sites, and the like. Myriad vectors for use in a wide variety of cells are known and these can be delivered using the disclosed compositions.

In some forms, the nucleic acids that are delivered to cells can contain expression controlling systems. For example, genes in vector systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

Useful promoters for controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway et al., Gene 18: 355-360 (1982)). Promoters from the host cell or related species can also be used.

Enhancer generally refers to a sequence of DNA that functions at no fixed distance from the transcription start site and can be either 5′ (Laimins et al., Proc. Natl. Acad. Sci. 78: 993 (1981)) or 3′ (Lusky et al., Mol. Cell Bio. 3: 1108 (1983)) to the transcription unit. Furthermore, enhancers can be within an intron (Baneji et al., Cell 33: 729 (1983)) as well as within the coding sequence itself (Osborne et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), enhancers from a eukaryotic cell virus can be used for general expression. Useful examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region can be active in all eukaryotic cell types, even if it is only expressed in a particular type of cell at a particular time. A useful promoter of this type is the CMV promoter (650 bases). Other useful promoters are SV40 promoters, cytomegalovirus (fall length promoter), and retroviral vector LTR.

Expression vectors used in eukaryotic host cells (yeast, fungi, insect, plant, animal, human or nucleated cells) can also contain sequences necessary for the termination of transcription. These regions are transcribed as polyadenylated segments in the untranslated portion of the mRNA encoding tissue factor protein. The 3′ untranslated regions also include transcription termination sites. It is preferred that the transcription unit also contain a polyadenylation region. One benefit of this region is that it increases the likelihood that the transcribed unit will be processed and transported like mRNA. The identification and use of polyadenylation signals in expression constructs is well established. Homologous polyadenylation signals can be used in the transgene constructs. The polyadenylation region can be derived from the SV40 early polyadenylation signal. It is also preferred that the transcribed units contain other standard sequences alone or in combination with the above sequences improve expression from, or stability of, the construct.

Vectors can include nucleic acid sequence encoding a marker product. This marker product can be used to determine if the gene has been delivered to the cell and once delivered is being expressed. Examples of useful marker genes are genes that encode β-galactosidase, luciferase, and green fluorescent protein.

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern and Berg, J. Molec. Appl. Genet. 1: 327 (1982)), mycophenolic acid, (Mulligan and Berg, Science 209: 1422 (1980)) or hygromycin, (Sugden et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin. Many other selectable marker genes are known for use in a variety of cell types.

Nucleic acid vaccines can also be delivered using the disclosed compositions. Nucleic acid vaccines are nucleic acids that encode one or more antigens. Delivery to, and expression by, cells in an animal can stimulate production of an immune response against the antigen(s). As used herein, a vaccine is any composition that is administered to a subject with the goal of establishing an immune response to a particular target or targets. In certain embodiments the vaccines will produce an immune response that is a protective immune response.

ii. Functional Nucleic Acids

Functional nucleic acids are nucleic acid molecules that have a specific function, such as binding a target molecule or catalyzing a specific reaction. Functional nucleic acid molecules can be divided into the following non-limiting categories. For example, functional nucleic acids include antisense molecules, aptamers, ribozymes, triplex-forming molecules, small interfering RNA, and external guide sequences. Functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Functional nucleic acids can be designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Small interfering RNA (siRNA) are nucleic acids molecules that mediate destruction of targeted RNA molecules in a cell. It is thought that siRNA involves a two-step mechanism for RNA interference (RNAi): an initiation step and an effector step. For example, in the first step, input double-stranded (ds) RNA (siRNA) is processed into small fragments, such as 21-23-nucleotide ‘guide sequences’. RNA amplification appears to be able to occur in whole animals. Typically then, the guide RNAs can be incorporated into a protein RNA complex which is cable of degrading RNA, the nuclease complex, which has been called the RNA-induced silencing complex (RISC). This RISC complex acts in the second effector step to destroy mRNAs that are recognized by the guide RNAs through base-pairing interactions. RNAi involves the introduction by any means of double stranded RNA into the cell which triggers events that cause the degradation of a target RNA. RNAi is a form of post-transcriptional gene silencing. For description of making and using RNAi molecules see, e.g., Hammond et al., Nature Rev Gen 2: 110-119 (2001); Sharp, Genes Dev 15: 485-490 (2001), Waterhouse et al., Proc. Natl. Acad. Sci. USA 95(23): 13959-13964 (1998) all of which are incorporated herein by reference in their entireties and at least for material related to delivery and maling of RNAi molecules.

RNAi has been shown to work in a number of cells, including mammalian cells. For work in mammalian cells it is preferred that the RNA molecules that will be used as targeting sequences within the RISC complex are shorter. For example, less than or equal to 50 or 40 or 30 or 29, 28, 27, 26, 25, 24, 23, 22, 21, 20, 19, 18, 17, 16, 15, 14, 13 , 12, 11, or 10 nucleotides in length. These RNA molecules can also have overhangs on the 3′ or 5′ ends relative to the target RNA which is to be cleaved. These overhangs can be at least or less than or equal to 1, 2, 3, 4, 5, 6, 7, 8, 9, 10, 15, or 20 nucleotides long. RNAi works in mammalian stem cells, such as mouse ES cells.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex-forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

iii. Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989,86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### E. Kits

The materials described above as well as other materials can be packaged together in any suitable combination as a kit useful for performing, or aiding in the performance of, the disclosed method: It is useful if the kit components in a given kit are designed and adapted for use together in the disclosed method. For example, disclosed are kits for delivering one or more compounds or compositions into cells, the kit comprising one or more aminoglycosides and one or more lipids. The kits also can contain buffers and/or solutions for forming and/or delivering the disclosed compositions. The disclosed kits can also include one or more targeting agents.

### F. Mixtures

Disclosed are mixtures formed by performing or preparing to perform the disclosed method. For example, disclosed are mixtures comprising one or more aminoglycosides and one or more lipids; one or more aminoglycosides, one or more lipids, and one or more cells; one or more aminoglycosides, one or more lipids, one or more compounds, and one or more cells; one or more aminoglycosides, one or more lipids, one or more compositions, and one or more cells; one or more aminoglycosides, one or more lipids, and one or more compounds; one or more aminoglycosides, one or more lipids, and one or more compositions; one or more aminoglycosides, one or more lipids, one or more compounds, and one or more compositions.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### G. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated.

### H. Data Structures and Computer Control

Disclosed are data structures used in, generated by, or generated from, the disclosed method. Data structures generally are any form of data, information, and/or objects collected, organized, stored, and/or embodied in a composition or medium. Measurements of the effects of the disclosed method stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Uses

The disclosed methods and compositions are applicable to numerous areas including, but not limited to, delivery of compounds and compositions to cells; delivery of compounds and compositions to cells in vitro; delivery of compounds and compositions to cells ex vivo; delivery of compounds and compositions to cells in vivo; administration of compounds and compositions to subjects; treatment of subjects; diagnosis of subjects; prognosis of subjects; detection and/or analysis of gene expression, genetic control, physiologic control, physiological state, signal transduction, and cell states in one or more cells. Other uses are disclosed, apparent from the disclosure, and/or will be understood by those in the art.

### Methods

Compounds and compositions can be delivered to cells using the disclosed compositions. In some forms of the method, compounds and compositions can be delivered into cells in vitro. In some forms of the method, compounds and compositions can be delivered into cells ex vivo. Such cells can be introduced into or administered to a subject. In some forms of the method, compounds and compositions can be delivered into cells in vivo. This can be accomplished by, for example, administering the disclosed compositions to a subject. Delivery of compounds and compositions into cells can be for any purpose. Generally, a given compound or composition can be delivered into a cell for a purpose related to the compound or composition, which purposes are generally known for a large number of compounds and compositions. For example, delivery of a vector into a cell using the disclosed compositions can be to obtain expression of the vector and/or stable transmission of the vector in progeny of the cell; delivery of a drug into a cell using the disclosed compositions can be to obtain an effect on the physiology of the cell by the drug (and thus an effect on the physiology of a subject if the cell is in or introduced to the subject); delivery of a siRNA or ribozyme into a cell using the disclosed compositions can be to obtain a change in, for example, gene expression or RNA processing by the siRNA or ribozyme. The purpose for delivery can be for any effect that the compound or composition can have or for which it was designed. Myriad compounds and compositions are known and they can be used with the disclosed compositions and methods for their known and expected purposed.

Disclosed are methods of treating subjects by administering the disclosed compositions to the subject. For example, compounds and compositions known, expected or suspected of having useful effects on a subject (such as therapeutic effects) can be used in the disclosed methods to treat subjects. Examples of compounds and compositions useful for this purpose include drugs, nucleic acids, and vectors. Also disclosed are methods of treating subjects by bringing into contact the disclosed compositions and cells and then administering the cells to the subject. Also disclosed are methods of administering compounds and compositions to subjects by administering the disclosed compositions to the subject, where the disclosed composition comprises the compound or composition to be delivered. Delivery can be, for example, non-specific, non-directed, non-targeted, specific, directed or targeted. As used herein, transfection refers to introduction of nucleic acids into a cell.

The disclosed methods generally can involve bringing into contact the disclosed composition and one or more cells. The composition can be maintained in contact with the cells for a sufficient time to allow the composition to be delivered into one or more of the cells. Any means or method can be used to bring the disclosed compositions into contact with cells. For delivery in vitro and ex vivo, plates, dishes, tubes, bottles, hoses, channels and the like can be used to hold or support cells and the disclosed compositions. For in vivo delivery, the disclosed compositions can be administered to a subject. Many modes and forms of administration can be used.

The disclosed compositions can be used as vaccines. For example, nucleic acid vaccines can also be delivered using the disclosed compositions. Vaccines can be, for example, prophylactic, that is, administered before a target is ever encountered, as is typically the case for Polio, measles, mumps, rubella, smallpox, chicken pox, and influenza vaccines, for example. Vaccines can also be therapeutic, providing an immune response to a target that is already within a subject, for example, a vaccine to a particular cancer. Typically vaccines are administered in a single or multiple doses called immunizations and are designed to generate memory T and B-cell populations.

### A. Administration to Subjects

The exact amount of the compositions to be used can vary from subject to subject, depending on the species, age, weight and general condition of the subject, the severity of the condition or disorder being treated, the particular compound or composition to be delivered, its mode of administration and the like. Thus, it is not possible to specify an exact amount for every composition. However, an appropriate amount can be determined by one of ordinary skill in the art using only routine experimentation given the teachings herein. Effective dosages and schedules for administering the disclosed compositions can be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products. A typical daily dosage of the disclosed composition can range from about 1 μg/kg to up to 100 mg/kg of body weight or more per day, depending on the factors mentioned above.

Following administration of a disclosed composition, the efficacy of the composition can be assessed in various ways well known to the skilled practitioner. Generally, the subject can be assessed for an intended effect or for a reduction or amelioration of one or more symptoms or effects of the condition treated.

The disclosed compositions can be administered in a number of ways depending on whether local or systemic treatment is desired, and on the area to be treated. Administration can be topical (including ophthalmical, vaginal, rectal, intranasal), oral, by inhalation, or parenteral, for example by intravenous drip, subcutaneous, intraperitoneal or intramuscular injection. The disclosed compositions can be administered intravenously, intraperitoneally, intramuscularly, subcutaneously, intracavity, or transdermally, including topical intranasal administration or administration by inhalant. As used herein, “topical intranasal administration” means delivery of the compositions into the nose and nasal passages through one or both of the names and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation.

### B. Targeting

The disclosed compositions can be targeted to particular cells or cell types via targeting agents such as antibodies, receptors, or receptor ligands. Examples of the use of targeting of specific proteins to tumor tissue are described in Senter et al., Bioconjugate Chem., 2:447-451, (1991); Bagshawe, Br. J. Cancer, 60:275-281, (1989); Bagshawe et al., Br. J. Cancer, 58:700-703, (1988); Senter et al., Bioconjugate Chem., 4:3-9, (1993); Battelli et al., Cancer Immunol. Inmunother., 35:421-425, (1992); Pietersz and McKenzie, Immunolog. Reviews, 129:57-80, (1992); and Roffler, et al., Biochem. Pharmacol, 42:2062-2065, (1991), Hughes et al., Cancer Research, 49:6214-6220, (1989); and Litzinger and Huang, Biochimica et Biophysica Acta, 1104:179-187, (1992).

In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)). Although receptors can be sued as targets for targeted the disclosed compositions to particular cells, the disclosed compositions do not require targeting via cell surface components to effectively deliver compounds and compositions to cells.

### C. Transgenic Animals and Targeted Gene Disruption

The disclosed compositions and methods can be used for targeted gene disruption and modification in any animal that can undergo these events. Gene modification and gene disruption refer to the methods, techniques, and compositions that surround the selective removal or alteration of a gene or stretch of chromosome in an animal, such as a mammal, in a way that propagates the modification through the germ line of the mammal. In general, a vector that is designed to homologously recombine with a region of a particular chromosome contained within a cell is delivered to the cell using the disclosed compositions. This homologous recombination event can produce a chromosome which has exogenous DNA introduced, for example in frame, with the surrounding DNA. This type of protocol allows for very specific mutations, such as point mutations, to be introduced into the genome contained within the cell.

Once the cell is produced through the methods described herein, an animal can be produced from this cell through either stem cell technology or cloning technology. For example, if the cell into which the nucleic acid was transfected was a stem cell for the organism, then this cell, after transfection and culturing, can be used to produce an organism which will contain the gene modification or disruption in germ line cells, which can then in turn be used to produce another animal that possesses the gene modification or disruption in all of its cells. In other methods for production of an animal containing the gene modification or disruption in all of its cells, cloning technologies can be used. These technologies generally take the nucleus of the transfected cell and either through fusion or replacement fuse the transfected nucleus with an oocyte which can then be manipulated to produce an animal. The advantage of procedures that use cloning instead of ES technology is that cells other than ES cells can be transfected. For example, a fibroblast cell, which is very easy to culture can be used as the cell which is transfected and has a gene modification or disruption event take place, and then cells derived from this cell can be used to clone a whole animal.

### EXAMPLE

### A. Enhanced Transfection With Neomycin/DOTAP Compositions

In this example, the effect of the aminoglycoside antibiotic neomycin on cationic lipid-mediated delivery of plasmid DNA and oligonucletides in cells is assessed. Neomycin can stabilize multiplex nucleic acid structures, such as DNA triplex, RNA triplex and other hybrid forms (Arya et al., J. Am. Chem. Soc. 2003, 125, 3733; Arya et al., J. Am. Chem. Soc. 2003, 125, 8070; Xue et al., Chemical Communications 2002, 70; Arya et al., J. Am. Chem. Soc. 2003, 125, 10148; Arya, in Top. Curr. Chem. DNA Binders, Editors-Chaires, J B; Waring, M; 2005, 253, 149; Arya et al., J. Am. Chem. Soc. 2001, 123, 5385; Arya et al., J. Am. Chem. Soc. 2001, 123, 11093; Arya and Coffee, Jr., Bioorg. Med. Chem. Lettrs, 2000, 10, 1897). This example explores the effect neomycin has on DNA transfection. The results indicate that neomycin, when combined with a cationic lipid preparation, like DOTAP, enhances transfection efficiency of both reporter plasmids and oligonucleotides and results in a significant increase in transgene expression. The enhancing effect of neomycin is mediated by increased uptake across the plasma membrane, although other mechanisms could favorably contribute to the phenomenon.

1. Materials and Methods

Cell culture. Human prostate cancer cells DU145 were grown in RPMI 1640 medium, supplemented with 10% heat-inactivated fetal bovine serum (Life Technologies) under standard conditions (Carbone et al., Nucleic Acids Res. 2003, 31, 833-843; Carbone et al., Nucleic Acids Res 2004, 32, 4358-4367).

Reporter plasmids. The reporter plasmid pGL3-Ets2 containing the firefly luciferase gene under control of Ets2 promoter has been previously described (Carbone et al., Nucleic Acids Res 2004, 32, 4358-4367). The pRL-SV40 reporter plasmid was purchased from Promega. The pEGFP plasmid was obtained from Clontech.

Oligonucleotides and other reagents. The fluorescein-conjugated phosphorothioate oligonucleotide (F-TFO) was purchased from Sigma. The oligonucleotide was purified by high-performance liquid chromatography (HPLC). The sequence of the F-TFO, which is identical to the Ets2-TFO, has been published previously (Carbone et al., Nucleic Acids Res. 2003, 31, 833-843). Stock solutions of oligonucleotides were made in sterile water. Oligonucleotide concentrations were determined with a spectrophotometer using appropriate nucleotide extinction coefficients. DOTAP (1,2-dioleoyl-3-trimethyl-ammonium-propane) was purchased from Roche (Indianapolis, Ind.). Neomycin was purchased from Sigma, and used as described previously (Arya et al., J. Am. Chem. Soc. 2003, 125, 3733-3744).

Luciferase reporter assay. DU145 cells were plated in 48-well plates at a density of 1.5×10⁴ cells/well. After 24 hours, cells were transfected with DOTAP-DNA complexes as previously described (Carbone et al., Nucleic Acids Res. 2003, 31, 833-843). Each transfection mixture contained a total of 2 μg of DNA, which was first diluted in 20 mM Hepes and mixed with appropriate amount of neomycin, and DOTAP at a 1:5 ratio. Mixtures of DNA-DOTAP and DNA-DOTAP-neomycin were allowed to sit at room temperature for 15 minutes for complex formation. The cells were then transfected by adding 200 μl of complexes to each well of the 48-well plate (200 ng of DNA/well). Cells were incubated with the transfection complexes for 5 hours, then the medium was removed and replaced with fresh medium. Cells were incubated for an additional 24 hours to allow expression of the reporter gene. Luciferase activity was measured with the Luciferase assay system from Promega.

EGFP reporter uptake. DU145 cells were plated in 6-well plates at a density of 1.5×10⁵ cells/well, grown overnight and then transfected. Trasfection mixtures contained 4 μg of pEGFP reporter plasmid with or without addition of neomycin and DOTAP at a 1:5 ratio as described above. Cells were incubated with the transfection complexes for 5 hours, then the medium was removed and replaced with fresh medium. After 24 hours, cells were harvested by centrifugation, recovered by centrifugation, washed once with PBS, and then analyzed using a FACSCalibur (Becton Dickinson). Data were analyzed using Cell Quest software.

Cellular uptake of oligonucleotides. Uptake of F-TFO and dauno-GT11 was examined 24 hours after transfection of DU145 cells (1.5×10⁵ cells/well in 6-well plates) with DOTAP with or without the addition of neomycin as described above. Concentration of F-TFO was 125 nM, corresponding to about 1 μg of DNA/well. After 24 hours, cells were washed and analyzed using a FACSCalibur as described above.

2. Results

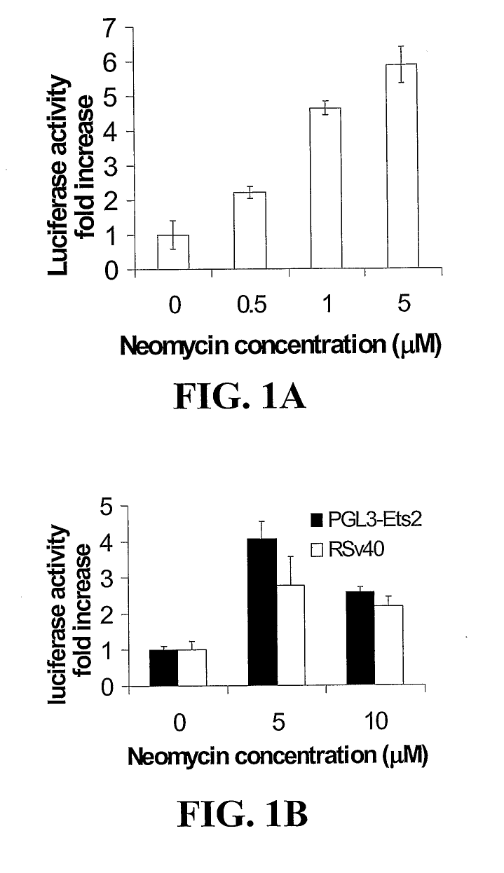

The effects of neomycin in combination with the cationic lipid preparation DOTAP on the uptake of luciferase reporter plasmids was determined. DU145 prostate cancer cells were transfected with the reporter plasmid pRL-SV40 using DOTAP in combination with increasing concentrations of neomycin. After 24 hours, cells were lysed and assayed for luciferase activity. The efficiency of transfection as assessed by the luciferase assay increased as the concentration of neomycin increased from 0.5 to 5 μM (FIG. 1A). Next, DU145 was transfected with the pGL3-Ets2 and pRL-SV40 reporter vectors in the presence of DOTAP, neomycin, or both DOTAP and neomycin. An increase in luciferase activity was observed from both reporter vectors when the plasmids were transfected with the combination of DOTAP and 5 μM neomycin compared to DOTAP alone (FIG. 1B). At 10 μM of neomycin the effect on luciferase activity was still evident (greater than 2-fold increase) but somewhat reduced compared to the lower concentration of neomycin, indicating that an optimal ratio between DOTAP, neomycin and DNA can be used to optimize results. Increased activity of the luciferase reporters in the presence of neomycin can be due to increased cellular uptake of plasmid DNA. No effect on luciferase reporter activity was observed when neomycin was added to the medium after the transfection, indicating that neomycin should be present in the transfection mix in order to achieve improve transfection efficiency.

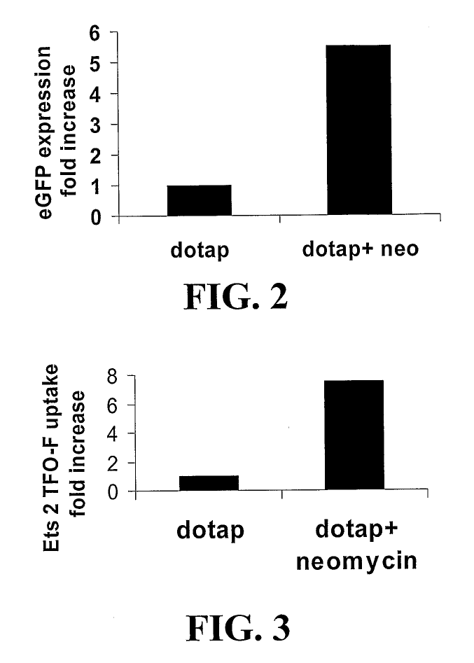

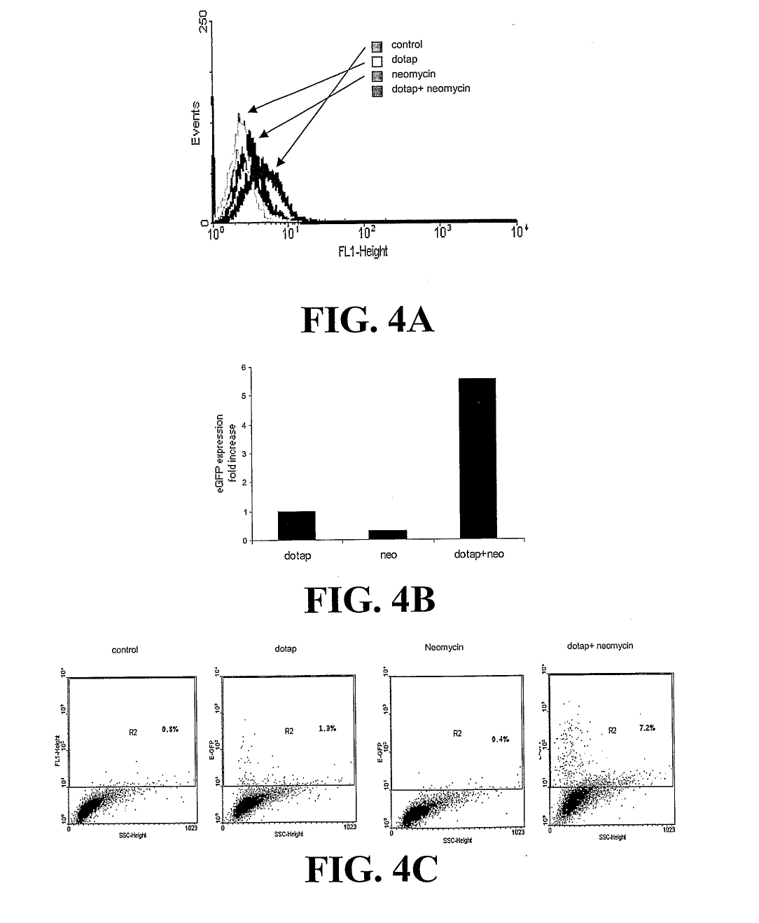

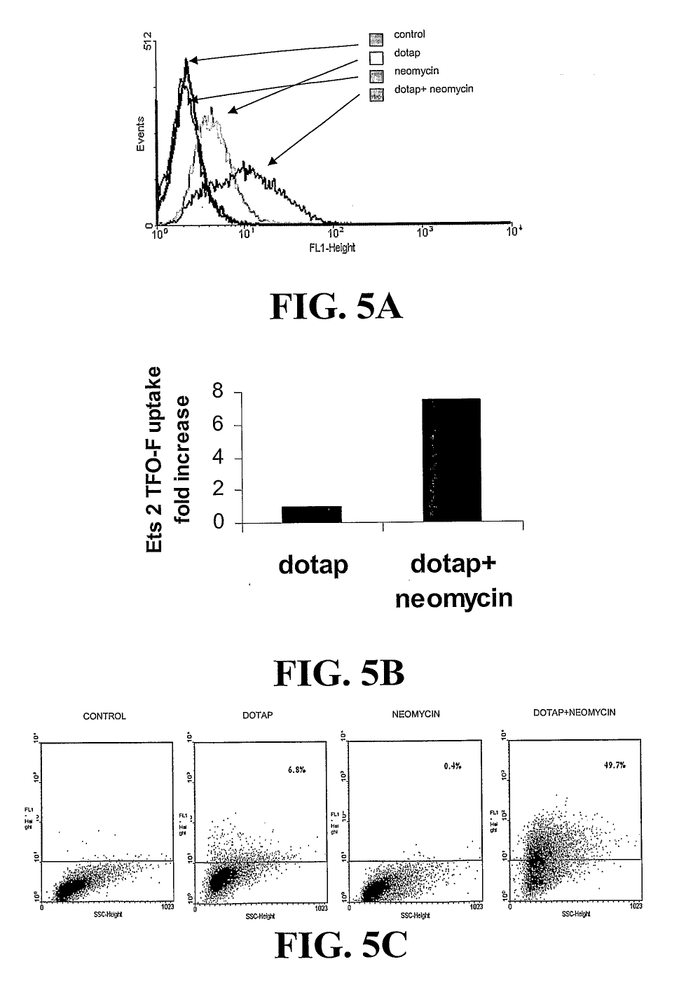

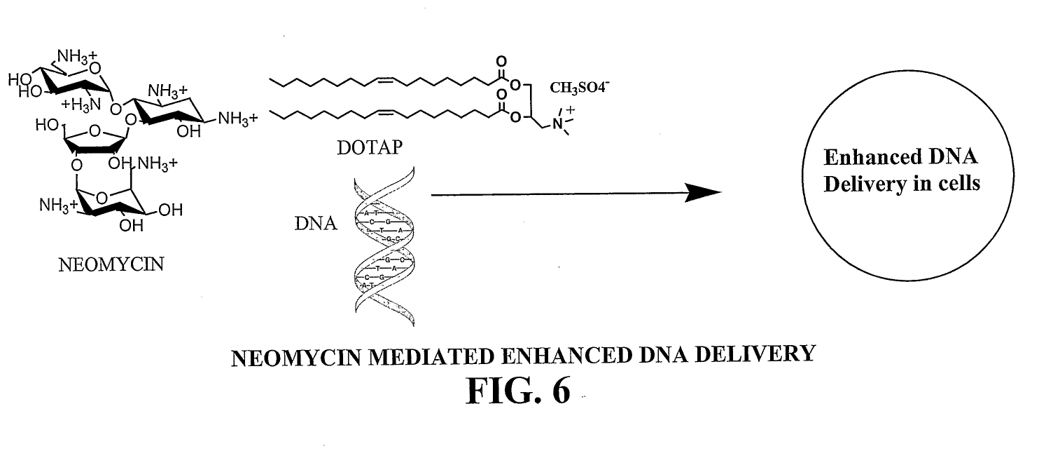

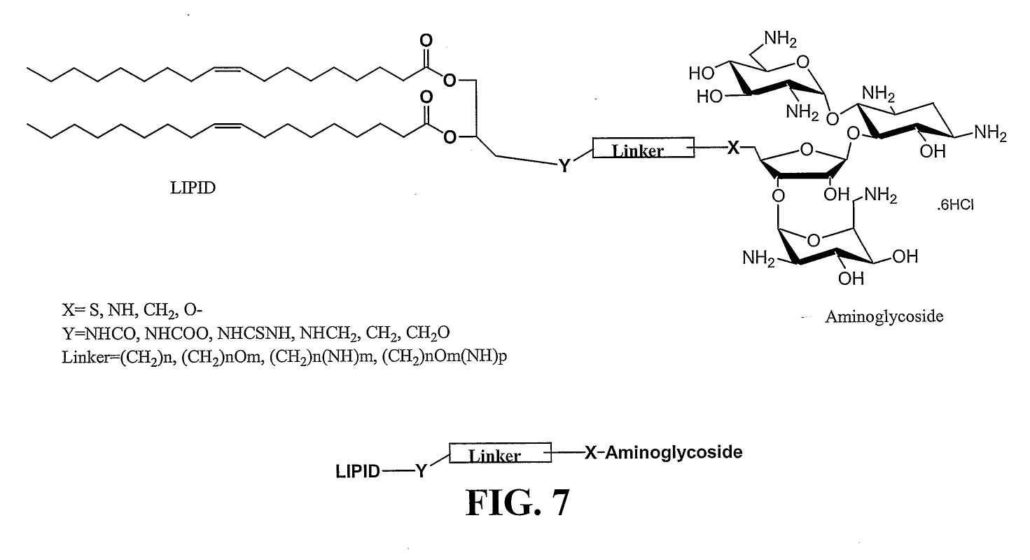

The effects of neomycin on transfection efficiency were further examined using another reporter system, a plasmid expressing green fluorescent protein, the pEGFP reporter vector. DU145 cells were transfected with the pEGFP plasmid in the presence of DOTAP, neomycin or both DOTAP and neomycin. After 24 hours, cells were collected and analyzed for EGFP expression by flow cytometry to examine both fluorescence intensity and percentage of EGFP-positive cells. There was a 5-fold increase in the number of EGFP-positive cells when DOTAP was combined with neomycin compared to DOTAP alone (FIGS. 2B and 2C). Neomycin alone marginally affected efficiency of DNA uptake. The mean fluorescence intensity was slightly higher in cells transfected with DOTAP and neomycin compared to DOTAP alone (mean fluorescent intensity: 31.9, 27.8 and 17.2 in cells transfected with DOTAP and neomycin, DOTAP alone, and without DOTAP, respectively).

Neomycin positively affected transfection efficiency of plasmid DNA mediated by DOTAP. This effect could be mediated by an increase in intracellular uptake and/or by an enhanced release of DNA from lipid complex into the cytoplasm. To determine whether neomycin had a similar effect on the uptake of oligonucleotides, efficiency of transfection of a fluorescein-labeled oligonucleotide was evaluated in the presence or absence of neomycin. For the tests shown in FIG. 3, a phosporothioate oligonucleotide directed to the Ets2 gene promoter (Ets2-TFO) was conjugated at the 5′ end with fluorescein (Carbone et al., Nucleic Acids Res. 2003, 31, 833). Cells were transfected with the fluorescein labeled TFO (F-TFO) for 6 hours and then incubated for 24 hours in fresh medium before the analysis by flow cytometry. At the concentration used in the test, only about 7% of cells were fluorescein-positive when transfected with DOTAP alone. The percentage of fluorescein-positive cells increased to about 50% when the oligonucleotide was delivered using the combination of DOTAP and neomycin. Therefore, neomycin enhanced uptake of the F-TFO by approximately 7-fold. Mean fluorescence intensity was also increased in cells transfected with DOTAP and neomycin compared to cells transfected with DOTAP alone (mean fluorescent intensity: 24.1, 19.4 and 17.6 in cells transfected with DOTAP and neomycin, DOTAP alone, and without DOTAP, respectively). Neomycin alone did not affect the oligonucleotide uptake (2.4% fluorescein-positive cells).

Thus, neomycin improves cationic lipid mediated transfection efficiency of reporter plasmids and intracellular uptake of oligonucleotides. These observations indicate that the increased reporter activity observed using the combination of DOTAP and neomycin is mediated by increased intracellular delivery of DNA. The adjuvant effect of neomycin may also be mediated by additional mechanisms, like facilitated release from lysosome and nuclear uptake (Lin et al., Biophysical Journal 2003, 84, 3307). Collectively, the data indicate that neomycin enhances DNA and oligonucleotide transfection efficiency mediated by cationic lipid reagents, like DOTAP. This effect is mediated at least in part by increased intracellular uptake.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a ”, “an”, and “the” include plural reference unless the context clearly dictates otherwise. Thus, for example, reference to “an amninoglycoside” includes a plurality of such aminoglycosides, reference to “the aminoglycoside” is a reference to one or more aminoglycosides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of delivery into a cell, the method comprising
bringing into contact one or more delivery compositions and one or more cells, wherein at least one of the delivery compositions comprises one or more aminoglycosides and one or more lipids, whereby at least one of the delivery compositions is taken into one or more of the cells.

2. The method of claim 1, wherein at least one of the aminoglycosides is covalently linked to at least one lipid.

3. The method of claim 2, wherein the aminoglycosides and lipid are covalently linked via a crosslinker.

4. The method of claim 1, wherein at least one of the delivery compositions further comprises one or more compounds or compositions to be delivered, wherein at least one of the compounds or compositions to be delivered is taken into one or more of the cells.

5. The method of claim 4, wherein at least one of the compounds to be delivered comprises nucleic acid.

6. The method of claim 4, wherein at least one of the compounds to be delivered comprises a vector, a gene, a functional nucleic acid, or a combination.

7. The method of claim 6, wherein the functional nucleic acid comprises an antisense molecule, aptamer, ribozyme, triplex forming molecule, small interfering RNA, nucleic acid vaccine, external guide sequence, or a combination.

8. The method of claim 6, wherein the gene encodes a heterologous protein or peptide.

9. The method of claim 4, wherein at least one of the compounds to be delivered comprises a drug or therapeutic agent.

10. The method of claim 1, wherein at least one of the cells is a prokaryotic cell or a eukaryotic cell.

11. The method of claim 1, wherein at least one of the cells is an animal cell.

12. The method of claim 1, wherein at least one of the cells is a mammalian cell.

13. The method of claim 1, wherein at least one of the cells is a human cell.

14. The method of claim 1, wherein the delivery compositions and cells are brought into contact in vitro, ex vivo or in vivo.

15. The method of claim 1, wherein the delivery compositions and cells are brought into contact by adding the delivery composition to a culture of the cells.

16. The method of claim 1, wherein at least one of the cells is administered to an animal.

17. The method of claim 1, wherein the delivery compositions and cells are brought into contact by administering the delivery compositions to an animal.

18. The method of claim 1, wherein at least one of the delivery compositions is targeted to at least one of the cells.

19. The method of claim 1, wherein at least one of the lipids is a cationic lipid.

20. The method of claim 1, wherein at least one of the lipids is a derivative of 1-amino-2,3-dihydroxypropane.

21. The method of claim 1, wherein at least one of the lipids comprises one of more fatty acids, wherein at least one of the fatty acids is capric acid (C10), lauric acid (C12), myristic acid (C14), palmitic acid (C16), margaric acid (C17), stearic acid (C18), arachidic acid (C20), behenic acid (C22), lignoceric acid (C24), cerotic acid (C26), montanic acid (C28), and melissic acid (C30), including branched and substituted derivatives thereof.

22. The method of claim 1, wherein at least one of the lipids is 1,2-dioleoyl-3-N,N,N-trimethylaminopropane chloride (DOTMA) or 1,2 bis(oleoyloxy)-3-(trimethylammonio) propane (DOTAP).

23. The method of claim 1, wherein at least one of the aminoglycosides is an antibiotic aminoglycoside.

24. The method of claim 1, wherein at least one of the aminoglycosides is neomycin.

25. The method of claim 1, wherein at least one of the aminoglycosides is a non-antibiotic aminoglycoside.

26. The method of claim 1, at least one of the aminoglycosides can interact with nucleic acids in the same manner as aminoglycoside antibiotics interact with nucleic acids.

27. The method of claim 1, wherein the ratio in at least one of the delivery compositions of at least one of the aminoglycosides and at least one of the lipids is about 1:100, 1:90, 1:80, 1:70, 1:60, 1:50, 1:45, 1:40, 1:35, 1:30, 1:25, 1:20, 1:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 60:1, 70:1, 80:1, 90:1, or 100:1.

28. The method of claim 1, wherein the ratio in at least one of the delivery compositions of at least one of the aminoglycosides and at least one of the lipids is about 1:5.

29. The method of claim 1, wherein the ratio in the delivery compositions of the aminoglycosides and the lipids is about 1:100, 1:90, 1:80, 1:70, 1:60, 1:50, 1:45, 1:40, 1:35, 1:30, 1:25, 1:20, 1:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 60:1, 70:1, 80:1, 90:1, or 100:1.

30. The method of claim 1, wherein the ratio in the delivery compositions of the aminoglycosides and the lipids is about 1:5.

31. A delivery composition comprising one or more aminoglycosides and one or more lipids.

32. The delivery composition of claim 31, wherein at least one of the aminoglycosides is covalently linked to at least one lipid.

33. The delivery composition of claim 32, wherein the aminoglycosides and lipid are covalently linked via a crosslinker.

34. The delivery composition of claim 31, wherein the delivery composition further comprises one or more compounds or compositions to be delivered.

35. The delivery composition of claim 34, wherein at least one of the compounds to be delivered comprises nucleic acid.

36. The delivery composition of claim 34, wherein at least one of the compounds to be delivered comprises a vector, a gene, a functional nucleic acid, or a combination.

37. The delivery composition of claim 36, wherein the functional nucleic acid comprises an antisense molecule, aptamer, ribozyme, triplex forming molecule, small interfering RNA, nucleic acid vaccine, external guide sequence, or a combination.

38. The delivery composition of claim 36, wherein the gene encodes a heterologous protein or peptide.

39. The delivery composition of claim 34, wherein at least one of the compounds to be delivered comprises a drug or therapeutic agent.

40. The delivery composition of claim 31, wherein the delivery composition is targeted to one or more cells.

41. The delivery composition of claim 31, wherein at least one of the lipids is a cationic lipid.

42. The delivery composition of claim 31, wherein at least one of the lipids is a derivative of 1-amino-2,3-dihydroxypropane.

43. The delivery composition of claim 31, wherein at least one of the lipids comprises one of more fatty acids, wherein at least one of the fatty acids is capric acid (C10), lauric acid (C12), myristic acid (C14), palmitic acid (C16), margaric acid (C17), stearic acid (C18), arachidic acid (C20), behenic acid (C22), lignoceric acid (C24), cerotic acid (C26), montanic acid (C28), and melissic acid (C30), including branched and substituted derivatives thereof.

44. The delivery composition of claim 31, wherein at least one of the lipids is 1,2-dioleoyl-3-N,N,N-trimethylaminopropane chloride (DOTMA) or 1,2 bis(oleoyloxy)-3-(trimethylammonio) propane (DOTAP).

45. The delivery composition of claim 31, wherein at least one of the aminoglycosides is an antibiotic aminoglycoside.

46. The delivery composition of claim 31, wherein at least one of the aminoglycosides is neomycin.

47. The delivery composition of claim 31, wherein at least one of the aminoglycosides is a non-antibiotic aminoglycoside.

48. The delivery composition of claim 31, at least one of the aminoglycosides can interact with nucleic acids in the same manner as aminoglycoside antibiotics interact with nucleic acids.

49. The delivery composition of claim 31, wherein the ratio in the delivery composition of at least one of the aminoglycosides and at least one of the lipids is about 1:100, 1:90, 1:80, 1:70, 1:60, 1:50, 1:45, 1:40, 1:35, 1:30, 1:25, 1:20, 1:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 60:1, 70:1, 80:1, 90:1, or 100:1.

50. The delivery composition of claim 31, wherein the ratio in the delivery composition of at least one of the aminoglycosides and at least one of the lipids is about 1:5.

51. A kit comprising one or more aminoglycosides, one or more lipids, and one or more compounds or compositions to be delivered into one or more cells.

52. The pharmaceutical composition of claim 51, wherein at least one of the aminoglycosides is covalently linked to at least one lipid.

53. The pharmaceutical composition of claim 52, wherein the aminoglycosides and lipid are covalently linked via a crosslinker.

54. The kit of claim 51, wherein at least one of the compounds to be delivered comprises nucleic acid.

55. The kit of claim 51, wherein at least one of the compounds to be delivered comprises a vector, a gene, a functional nucleic acid, or a combination.

56. The kit of claim 55, wherein the functional nucleic acid comprises an antisense molecule, aptamer, ribozyme, triplex forming molecule, small interfering RNA, nucleic acid vaccine, external guide sequence, or a combination.

57. The kit of claim 55, wherein the gene encodes a heterologous protein or peptide.

58. The kit of claim 51, wherein at least one of the compounds to be delivered comprises a drug or therapeutic agent.

59. The kit of claim 51, wherein at least one of the cells is a prokaryotic cell or a eukaryotic cell.

60. The kit of claim 51, wherein at least one of the cells is an animal cell.

61. The kit of claim 51, wherein at least one of the cells is a mammalian cell.

62. The kit of claim 51, wherein at least one of the cells is a human cell.

63. The kit of claim 51, wherein at least one of the delivery compositions is targeted to at least one of the cells.

64. The kit of claim 51, wherein at least one of the lipids is a cationic lipid.

65. The kit of claim 51, wherein at least one of the lipids is a derivative of 1-amino-2,3-dihydroxypropane.

66. The of claim 51, wherein at least one of the lipids comprises one of more fatty acids, wherein at least one of the fatty acids is capric acid (C10), lauric acid (C12), myristic acid (C14), palmitic acid (C16), margaric acid (C17), stearic acid (C18), arachidic acid (C20), behenic acid (C22), lignoceric acid (C24), cerotic acid (C26), montanic acid (C28), and melissic acid (C30), including branched and substituted derivatives thereof.

67. The kit of claim 51, wherein at least one of the lipids is 1,2-dioleoyl-3-N,N,N-trimethylaminopropane chloride (DOTMA) or 1,2 bis(oleoyloxy)-3-(trimethylammonio) propane (DOTAP).

68. The kit of claim 51, wherein at least one of the aminoglycosides is an antibiotic aminoglycoside.

69. The kit of claim 51, wherein at least one of the aminoglycosides is neomycin.

70. The kit of claim 51, wherein at least one of the aminoglycosides is a non-antibiotic aminoglycoside.

71. The kit of claim 51, at least one of the aminoglycosides can interact with nucleic acids in the same manner as aminoglycoside antibiotics interact with nucleic acids.

72. The kit of claim 51, wherein at least one of the compounds to be delivered comprises a vector, wherein the vector can be engineered to comprise one or more nucleic acid sequences of interest.

73. The kit of claim 51, wherein at least one or more aminoglycosides, at least one of the lipids, and at least one of the compounds or compositions to be delivered are formulated as a delivery composition.

74. A pharmaceutical composition comprising one or more aminoglycosides, one or more lipids, and one or more pharmaceutical compounds or compositions.

75. The pharmaceutical composition of claim 74, wherein at least one of the aminoglycosides is covalently linked to at least one lipid.

76. The pharmaceutical composition of claim 75, wherein the aminoglycosides and lipid are covalently linked via a crosslinker.

77. The pharmaceutical composition of claim 74, wherein at least one of the compounds to be delivered comprises a vector, a gene, a functional nucleic acid, or a combination.

78. The pharmaceutical composition of claim 77, wherein the functional nucleic acid comprises an antisense molecule, aptamer, ribozyme, triplex forming molecule, small interfering RNA, nucleic acid vaccine, external guide sequence, or a combination.

79. The pharmaceutical composition of claim 77, wherein the gene encodes a heterologous protein or peptide.

80. The pharmaceutical composition of claim 74, wherein at least one of the compounds to be delivered comprises a drug or therapeutic agent.

81. The pharmaceutical composition of claim 74, wherein the pharmaceutical composition is targeted to one or more cells.

82. The pharmaceutical composition of claim 74, wherein at least one of the lipids is a cationic lipid.

83. The pharmaceutical composition of claim 74, wherein at least one of the lipids is a derivative of 1-amino-2,3-dihydroxypropane.

84. The pharmaceutical composition of claim 74, wherein at least one of the lipids comprises one of more fatty acids, wherein at least one of the fatty acids is capric acid (C10), lauric acid (C12), myristic acid (C14), palmitic acid (C16), margaric acid (C17), stearic acid (C18), arachidic acid (C20), behenic acid (C22), lignoceric acid (C24), cerotic acid (C26), montanic acid (C28), and melissic acid (C30), including branched and substituted derivatives thereof.

85. The pharmaceutical composition of claim 74, wherein at least one of the lipids is 1,2-dioleoyl-3-N,N,N-trimethylaminopropane chloride (DOTMA) or 1,2 bis(oleoyloxy)-3-(trimethylammonio) propane (DOTAP).

86. The pharmaceutical composition of claim 74, wherein at least one of the aminoglycosides is an antibiotic aminoglycoside.

87. The pharmaceutical composition of claim 74, wherein at least one of the aminoglycosides is neomycin.

88. The pharmaceutical composition of claim 74, wherein at least one of the aminoglycosides is a non-antibiotic aminoglycoside.

89. The pharmaceutical composition of claim 74, at least one of the aminoglycosides can interact with nucleic acids in the same manner as aminoglycoside antibiotics interact with nucleic acids.

90. The pharmaceutical composition of claim 74, wherein the ratio in the pharmaceutical composition of at least one of the aminoglycosides and at least one of the lipids is about 1:100, 1:90, 1:80, 1:70, 1:60, 1:50, 1:45, 1:40, 1:35, 1:30, 1:25, 1:20, 1:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 60:1, 70:1, 80:1, 90:1, or 100:1.

91. The pharmaceutical composition of claim 74, wherein the ratio in the pharmaceutical composition of at least one of the aminoglycosides and at least one of the lipids is about 1:5.
